(19) United States
(12) Patent Application Publication
(10) Pub. No.: US 2016/0083754 A1
(43) Pub. Date: Mar. 24, 2016

(54) PROCESSING BIOMASS

(71) Applicant: XYLECO, INC., Woburn, MA (US)

(72) Inventors: Marshall MEDOFF, Brookline, MA (US); Thomas Craig MASTERMAN, Rockport, MA (US); Jaewoong MOON, Andover, MA (US)

(73) Assignee: Xyleco, Inc., Woburn, MA (US)

(21) Appl. No.: 14/891,507

(22) PCT Filed: May 16, 2014
(86) PCT No.: PCT/US14/38316

§ 371 Date: Nov. 16, 2015

Related U.S. Application Data
(60) Provisional application No. 61/941,771, filed on Feb. 19, 2014.
(60) Provisional application No. 61/824,582, filed on May 17, 2013.
(60) Provisional application No. 61/824,597, filed on May 17, 2013.

(51) Int. Cl. C12P 7/46

(52) U.S. Cl. CPC C12P 7/46

(57) ABSTRACT

Biomass (e.g., plant biomass, animal biomass, and municipal waste biomass) is processed to produce useful intermediates and products, such as poly carboxylic acids and poly carboxylic acid derivatives.

### CROSS REFERENCE TO RELATED APPLICATIONS

This application claims priority from the following provisional applications: U.S. Ser. No. 61/824,582, filed May 17, 2013, U.S. Ser. No. 61/824,597, filed May 17, 2013 and 61/941,771, filed Feb. 19, 2014. The full disclosure of each of these provisional applications is incorporated by reference herein.

### BACKGROUND OF THE INVENTION

Many potential lignocellulosic feedstocks are available today, including agricultural residues, woody biomass, municipal waste, oilseeds/cakes and seaweed, to name a few. At present, these materials are often under-utilized, being used, for example, as animal feed, biocompost materials, burned in a co-generation facility or even landfilled.

Lignocellulosic biomass includes crystalline cellulose fibrils embedded in a hemicellulose matrix, surrounded by lignin. This produces a compact matrix that is difficult to access by enzymes and other chemical, biochemical and/or biological processes. Cellulosic biomass materials (e.g., biomass material from which the lignin has been removed) is more accessible to enzymes and other conversion processes, but even so, naturally-occurring cellulosic materials often have low yields (relative to theoretical yields) when contacted with hydrolyzing enzymes. Lignocellulosic biomass is even more recalcitrant to enzyme attack. Furthermore, each type of lignocellulosic biomass has its own specific composition of cellulose, hemicellulose and lignin.

### SUMMARY

Generally, this disclosure relates to treating a reduced recalcitrance biomass material (e.g., cellulosic, lignocellulosic and/or starchy materials) with one or more enzymes and/or one or more organisms (e.g., in any order) to produce a poly carboxylic acid, such as a di-, tri- or tetracarboxylic acid. For example, polycarboxylic acids selected from the group consisting of oxalic acid, malonic acid, succinic acid, tartaric acid, glutaric acid, adipic acid, pimelic acid, suberic acid, azelaic acid, sebacic acid, undecanedioic acid, dodecanedioic acid, maleic acid, fumaric acid, glutaconic acid, traumatic acid, muconic acid, phthalic acid, isophthalic acid, terephthalic acid, citric acid, isocitric acid, aconitic acid, mellitic acid and mixtures of these. For example, a biomass material having reduced recalcitrance can be treated with one of more enzymes to liberate one or more sugars and then one or more of the liberated sugars can be fermented to one of more polycarboxylic acids, such as succinic acid. The inventions herein also relate to methods, processes and systems for converting a material, such as a biomass feedstock, e.g., cellulosic, starchy or lignocellulosic materials, to useful products, for example, derivatives of poly carboxylic acids.

In one aspect, the invention features methods for converting poly carboxylic acids to a product. The poly carboxylic acids can be made by treating a reduced recalcitrance lignocellulosic or cellulosic material with one or more enzymes. Optionally, the treatments to produce the reduced recalcitrance lignocellulosic or cellulosic material can include at least one of irradiation, sonication, oxidation, pyrolysis and steam explosion to produce the reduced recalcitrance material. For example, an irradiation treatment (e.g., with a dose of between about 20 and 50 Mrad) and can be used to reduce the recalcitrance of the material. The radiation treatment can include an electron beam irradiation. Optionally, treating is performed initially with one of more enzymes to release one or more sugars from the lignocellulosic or cellulosic material followed by one or more organisms to produce the poly carboxylic acid. For example, the sugars can be selected from the group consisting of glucose, xylose, sucrose, maltose, lactose, mannose, galactose, arabinose, fructose, disaccharides of any one or two of these (e.g., cellobiose or fructose), cellobiose, sucrose, poly saccharides of any two or more of these, and mixtures of these. In some implementations, treating converts one or more of the sugars to an intermediate product (e.g., ethanol or glycol by fermentation of the sugar) prior to conversion to the poly carboxylic acid.

In some implementations, the poly carboxylic acid is chemically converted to the product. For example, by reactions including polymerization, condensations, isomerization, esterification, alkylation, oxidation, amination, acid halide formation (e.g., acid chloride or acid bromide formation from the acid or anhydride), reduction, hydrogenation, cyclization, ion exchange, anhydration, acylation and combinations thereof. Optionally, the chemically converting steps can include catalytic conversion, non-catalytic conversion and combinations thereof.

In some implementations the poly carboxylic acid is succinic acid and the product it is converted to includes tetrahydrofuran, gamma-butyro lactone, 2-pyrrolidinone, N-methyl-2-pyrrolidinone (NMP), N-viny-2-pyrrolidinone, succinimide, N-hydroxysuccinimide, succindiamide, succinyl chloride, succinic acid anhydride, maleic anhydride, 1,4-diaminobutane, succinonitrile, 1,4-butandiol, dimethyl succinate or mixtures of these.

In another aspect, the invention features a method of making a product, where the method includes contacting a mixed sugar solution that has a nitrogen source (e.g., yeast extract) and inorganic salts (e.g., any one or more of NaH₂PO₄, Na₂HPO₄, NaCl, MgCl₂ and CaCl₂) with a succinic acid producing organism to produce succinic acid. For example, the succinic acid producing organism ferments at least one of the sugars in the sugar solution to succinic acid. The method further includes purifying the succinic acid and converting (e.g., chemically converting) the purified succinic acid to the product. The sugar solution can be made by saccharifying an electron beam treated cellulosic or lignocellulosic biomass. For example, the cellulosic or lignocellulosic material receives a dose of between about 10 and about 50 Mrad of radiation. In some implementations, the organism is selected from the group consisting of Actinobacillus succinogenes, Anaerobiospirillum succiniciproducens, Mannheimia succiniciproducens and, PEP carboxylase over-expressing E. coli.

In another aspect, the invention features a method for mating a product including treating a reduced recalcitrance lignocellulosic or cellulosic material with one or more enzymes and/or organisms to produce a poly carboxylic acid. Optionally, a feedstock (e.g., a cellulosic or lingnocellulosic material) is pretreated with ionizing radiation to produce the reduced recalcitrance of the lignocellulosic or cellulosic material. Optionally, treating the reduced recalcitrance feedstock includes treating initially with one of more enzymes to release one or more sugar from the lignocellulosic or cellulosic material followed by one or more organisms to produce the poly carboxylic acid. Optionally, the poly carboxylic acid is not succinic acid.

Products described herein, for example, succinic acid and succinic acid derivatives, e.g., acyl derivatives and anhydride derivatives, can be produced by methods described herein. Fermentative methods and/or combinations of fermentative methods and chemical methods can be very efficient, providing high biomass conversion, selective conversion and high production rates. The methods describe herein are also advantageous in that the starting materials (e.g., sugars and/or alcohols) can be completely derived from biomass (e.g., cellulosic and lignocellulosic materials). In addition, some of the fermentative technologies described can also adsorb additional CO₂ since some fermenting species proceed through a CO₂ fixing mechanism. Some of the products described herein, such as biopolymers, are compostable, biodegradable and/or recyclable. Therefore, the methods described herein can provide useful materials and products from renewable sources (e.g., biomass), sequester carbon and the products themselves can be re-utilized or simply safely returned to the environment.

Implementations of the invention can optionally include one or more of the following summarized features. In some implementations, the selected features can be applied or utilized in any order while in other implementations a specific selected sequence is applied or utilized. Individual features can be applied or utilized more than once in any sequence and even continuously. In addition, an entire sequence, or a portion of a sequence, of applied or utilized features can be applied or utilized once, repeatedly or continuously in any order. In some optional implementations, the features can be applied or utilized with different, or where applicable the same, set or varied, quantitative or qualitative parameters as determined by a person skilled in the art. For example, parameters of the features such as size, individual dimensions (e.g., length, width, height), location of, degree (e.g., to what extent such as the degree of recalcitrance), duration, frequency of use, density, concentration, intensity and speed can be varied or set, where applicable, as determined by a person of skill in the art.

Features, for example, include: A method for making a product; treating a reduced recalcitrance lignocellulosic or cellulosic material with one or more enzymes and/or organisms to produce a poly carboxylic acid; converting a poly carboxylic acid to a product; pretreating a feedstock with at least irradiation to produce the reduced recalcitrance lignocellulosic or cellulosic material; pretreating a feedstock with at least sonication to produce the reduced recalcitrance lignocellulosic or cellulosic material; pretreating a feedstock with at least oxidation to produce the reduced recalcitrance lignocellulosic or cellulosic material; pretreating a feedstock with at least pyrolysis to produce the reduced recalcitrance lignocellulosic or cellulosic material; pretreating a feedstock with at least steam explosion to produce the reduced recalcitrance lignocellulosic or cellulosic material; pretreating a feedstock with at least electron beam irradiation to produce the reduced recalcitrance lignocellulosic or cellulosic material; converting a poly carboxylic acid to a product comprises chemically converting; chemically converting includes at least polymerization; chemically converting includes at least condensations; chemically converting includes at least isomerization; chemically converting includes at least esterification; chemically converting includes at least alkylation; chemically converting includes at least oxidation; chemically converting includes at least amination; chemically converting includes at least acid halide formation; chemically converting includes at least reduction; chemically converting includes at least hydrogenation; chemically converting includes at least cyclization; chemically converting includes at least ion exchange; chemically converting includes at least anhydration; chemically converting includes at least acylation; chemically converting includes catalytic conversion; chemically converting includes non-catalytic conversion; treating is performed with one of more enzymes to release one or more sugar from a lignocellulosic or cellulosic material followed by one or more organisms to produce the poly carboxylic acid; a sugar released from lignocellulosic or cellulosic material is of glucose; a sugar released from lignocellulosic or cellulosic material is of xylose; a sugar released from lignocellulosic or cellulosic material is sucrose; a sugar released from lignocellulosic or cellulosic material is of maltose; a sugar released from lignocellulosic or cellulosic material is lactose; a sugar released from lignocellulosic or cellulosic material is mannose; a sugar released from lignocellulosic or cellulosic material is of galactose; a sugar released from lignocellulosic or cellulosic material is arabinose; a sugar released from lignocellulosic or cellulosic material is of fructose; a sugar released from lignocellulosic or cellulosic material is a disaccharide that includes at least one or include two of, glucose, xylose, maltose, lactose, mannose, galactose, arabinose or fructose; a sugar released from lignocellulosic or cellulosic material is cellobiose; a sugar released from lignocellulosic or cellulosic material is sucrose; a sugar released from lignocellulosic or cellulosic material is a poly saccharides that includes any of two or more of glucose, xylose, maltose, lactose, mannose, galactose, arabinose or fructose; a sugar released from lignocellulosic or cellulosic material is cellobiose; treating converts one or more sugars to an intermediate product prior to conversion to a poly carboxylic acid; an intermediate product is ethanol; an intermediate product is glycol; a sugar is converted to an intermediate product by fermentation; a poly carboxylic acid is oxalic acid; a poly carboxylic acid is malonic acid; a poly carboxylic acid is succinic acid; a poly carboxylic acid is tartaric acid; a poly carboxylic acid is glutaric acid; a poly carboxylic acid is adipic acid; a poly carboxylic acid is pimelic acid; a poly carboxylic acid is suberic acid; a poly carboxylic acid is azelaic acid; a poly carboxylic acid is sebacic acid; a poly carboxylic acid is undecanedioic acid; a poly carboxylic acid is dodecanedioic acid; a poly carboxylic acid is maleic acid; a poly carboxylic acid is fumaric acid; a poly carboxylic acid is glutaconic acid; a poly carboxylic acid is traumatic acid; a poly carboxylic acid is muconic acid; a poly carboxylic acid is phthalic acid; a poly carboxylic acid is isophthalic acid; a poly carboxylic acid is terephthalic acid; a poly carboxylic acid is citric acid; a poly carboxylic acid is isocitric acid; a poly carboxylic acid is aconitic acid; a poly carboxylic acid is mellitic acid; a product made from a dicarboxylic acid is tetrahydrofuran; a product made from a dicarboxylic acid is gamma-butyro lactone; a product made from a dicarboxylic acid is 2-pyrrolidinone; a product made from a dicarboxylic acid is N-methyl-2-pyrrolidinone (NMP); a product made from a dicarboxylic acid is N-viny-2-pyrrolidinone; a product made from a dicarboxylic acid is succinimide; a product made from a dicarboxylic acid is N-hydroxysuccinimide; a product made from a dicarboxylic acid is succindiamide; a product made from a dicarboxylic acid is succinyl chloride; a product made from a dicarboxylic acid is succinic acid anhydride; a product made from a dicarboxylic acid is maleic anhydride; a product made from a dicarboxylic acid is 1,4-diaminobutane, succinonitrile; a product made from a dicarboxylic acid is 1,4-butandiol and dimethyl succinate

Features, for example, an include or further include: a method for making a product; contacting a mixed sugar solution comprising a nitrogen source and inorganic salts with a succinic acid producing organism to produce succinic acid; purifying a succinic acid; converting a purified succinic acid to a product; a sugar solution is made by saccharifying an electron beam treated cellulosic or lignocellulosic biomass; an inorganic salt includes NaH₂PO₄; an inorganic salt includes Na₂HPO₄; an inorganic salt includes NaCl; an inorganic salt includes MgCl₂; an inorganic salt includes CaCl₂; a nitrogen source includes yeast extract; an organism is Actinobacillus succinogenes, Anaerobiospirillum succiniciproducens; an organism is Mannheimia succiniciproducens; an organism is PEP carboxylase over-expressing E. coli; converting include chemically converting; a cellulosic or lignocellulosic material receives between about 10 and about 50 Mrad of radiation.

Other features, for example, an include or further include: a method for making a product; treating a reduced recalcitrance lignocellulosic or cellulosic material with one or more enzymes and/or organisms to produce a poly carboxylic acid; a feedstock is pretreated with ionizing radiation to produce a reduced recalcitrance lignocellulosic or cellulosic material; ionizing radiation is an electron beam; treating is performed initially with one of more enzymes to release one or more sugar from a lignocellulosic or cellulosic material followed by one or more organisms to produce to poly carboxylic acid; a produced poly carboxylic acid is oxalic acid; a produced poly carboxylic acid is malonic acid; a produced poly carboxylic acid is succinic acid; a produced poly carboxylic acid is tartaric acid; a produced poly carboxylic acid is glutaric acid; a produced poly carboxylic acid is adipic acid; a produced poly carboxylic acid is pimelic acid; a produced poly carboxylic acid is suberic acid; a produced poly carboxylic acid is azelaic acid; a produced poly carboxylic acid is sebacic acid; a produced poly carboxylic acid is undecanedioic acid; a produced poly carboxylic acid is dodecanedioic acid; a produced poly carboxylic acid is maleic acid; a produced poly carboxylic acid is fumaric acid; a produced poly carboxylic acid is glutaconic acid; a produced poly carboxylic acid is traumatic acid; a produced poly carboxylic acid is muconic; a produced poly carboxylic acid is acid, phthalic acid; a produced poly carboxylic acid is isophthalic acid; a produced poly carboxylic acid is terephthalic acid; a produced poly carboxylic acid is citric acid; a produced poly carboxylic acid is isocitric acid; a produced poly carboxylic acid is aconitic acid; a produced poly carboxylic acid is mellitic acid.

Other features and advantages of the invention will be apparent from the following detailed description, and from the claims.

### DESCRIPTION OF THE DRAWING

The foregoing will be apparent from the following more particular description of example embodiments of the invention, as illustrated in the accompanying. The drawings are not necessarily to scale, emphasis instead being placed upon illustrating embodiments of the present invention.

FIG. 1 is a flow diagram showing processes for manufacturing products from a biomass feedstock.

FIG. 2 shows the chemical structures of some poly carboxylic acids.

FIG. 3 is a schematic showing a biochemical pathway for the fermentation of sugars to succinic acid.

FIG. 4A is a schematic showing some possible chemical pathways for producing succinic acid derived products. FIG. 4B shows some reactions for a derivative of succinic acid.

FIG. 5 is a schematic view of a reaction system for the polymerization of monomers.

FIG. 6A is a top view of a first embodiment of a reciprocating scraper. FIG. 6B is a front cut-out view of the first embodiment of a reciprocating scraper. FIG. 6C is a top view of a second embodiment of a reciprocating scraper. FIG. 6D is a front cut-out view of the second embodiment of a reciprocating scraper.

FIG. 7A shows a schematic of a polymerization unit that has an example of a thin film polymerization/devolatilization device and an extruder. FIG. 7B shows a cutaway of the thin film polymerization/devolatilization device with the sloped surface upon which the molten polymer flows.

FIG. 8A shows a small scale polymerization unit that has an example of a laboratory-scale thin film polymerization/devolatilization device. FIG. 8B shows a cutaway of the thin film polymerization/devolatilization device with the sloped surface upon which the molten polymer flows.

FIG. 9 is a schematic showing the preparation of glucoside-based gemini surfactants.

FIG. 10 is a schematic showing the preparation of non-ionic surfactants using di-carboxylic acids.

FIG. 11 is a plot showing the consumption of glucose and production of succinic acid.

FIG. 12 is a plot showing the consumption of xylose and production of succinic acid

FIG. 13 is a plot showing the consumption of glucose+xylose and production of succinic acid.

FIG. 14 is a plot of sugars consumed and products produced using a 1.2 L Bioreactor culture of Actinobacillus succinogenes.

### DETAILED DESCRIPTION

Using the equipment, methods and systems described herein, cellulosic and lignocellulosic feedstock materials, For example, that can be sourced from biomass (e.g., plant biomass, animal biomass, paper, and municipal waste biomass), can be turned into useful products and intermediates such as sugars and poly carboxylic acids. Included are equipment, methods and systems to chemically convert the primary products produced from the biomass to secondary product such as polymers (e.g., polyesters and poly urethanes), polymer derivatives (e.g., composites, elastomers and co-polymers), solvents (e.g., tetrahydrofuran, N-methyl-2-pyrollidone), pharmaceuticals and other useful products.

Biomass is a complex feedstock. For example, lignocellulosic materials include different combinations of cellulose, hemicellulose and lignin. Cellulose is a linear polymer of glucose. Hemicellulose is any of several heteropolymers, such as xylan, glucuronoxylan, arabinoxylans and xyloglucan. The primary sugar monomer present (e.g., present in the largest concentration) in hemicellulose is xylose, although other monomers such as mannose, galactose, rhamnose, arabinose and glucose are present. Although all lignins show variation in their composition, they have been described as an amorphous dendritic network polymer of phenyl propene units. The amounts of cellulose, hemicellulose and lignin in a specific biomass material depends on the source of the biomass material. For example, wood-derived biomass can be about 38-49% cellulose, 7-26% hemicellulose and 23-34% lignin depending on the type. Grasses typically are 33-38% cellulose, 24-32% hemicellulose and 17-22% lignin. Clearly lignocellulosic biomass constitutes a large class of substrates.

Enzymes and biomass-destroying organisms that break down biomass, such as the cellulose, hemicellulose and/or the lignin portions of the biomass as described above, contain or manufacture various cellulolytic enzymes (cellulases), ligninases, xylanases, hemicellulases or various small molecule biomass-destroying metabolites. A cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally cellobiase cleaves cellobiose to yield glucose. In the case of hemicellulose, a xylanase (e.g., hemicellulase) acts on this biopolymer and releases xylose as one of the possible products.

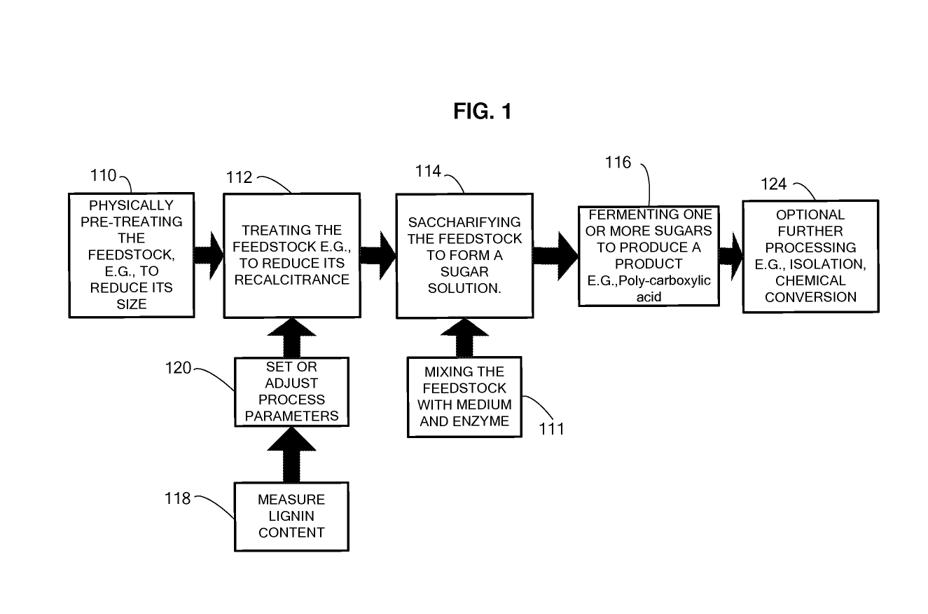

FIG. 1 is a flow diagram showing processes for manufacturing poly-carboxylic acids (e.g., cellulosic or lignocellulosic materials) and further converting the acid to another product. In an initial step 110 the method includes optionally mechanically treating a cellulosic and/or lignocellulosic feedstock, For example, to comminute/size reduce the feedstock. Before and/or after this treatment, the feedstock can be treated with another physical treatment 112, For example, irradiation, sonication, steam explosion, oxidation, pyrolysis or combinations of these, to reduce or further reduce its recalcitrance. A sugar solution e.g., including glucose and/or xylose, is formed by saccharifying the feedstock 114. The saccharification can be, for example, accomplished efficiently by, in any order and optionally repeatedly, the addition of one or more enzymes e.g., cellulases and/or xylanases 111, heating and/or one or more acids. A product or several products can be derived from the sugar solution, for example, by fermentation to a poly carboxylic acid 116. Following fermentation, the fermentation product (e.g., or products, or a subset of the fermentation products) can be purified or they can be further processed. For example, chemically converted (e.g., reduced, oxidized, undergo atom substitution reactions such as aminations, cyclized, polymerized or combinations of these) and/or isolated 124. In some embodiments, the sugar solution is a mixture of sugars and the organism ferments two or more of the sugars. Optionally the sugar solution is a mixture of sugars and the organism selectively ferments only one of the sugars. The fermentation of only one of the sugars in a mixture can be advantageous as described in PCT Application No. PCT/US14/21813 filed Mar. 7, 2014, the entire disclosure of which is incorporated herein by reference. If desired, the steps of measuring lignin content 118 and setting or adjusting process parameters based on this measurement 120 can be performed at various stages of the process, for example, as described in U.S. Pat. No. 8,415,122, issued Apr. 9, 2013 the entire disclosure of which is incorporated herein by reference. Optionally, enzymes (e.g., in addition to cellulases and xylanases) can be added in step 114, For example, a glucose isomerase can be used to isomerize glucose to fructose. Some relevant uses of isomerase are discussed in PCT Application No. PCT/US12/71093, filed on Dec. 20, 2012, the entire disclosure of which is incorporated herein by reference.

In some embodiments the liquids after saccharification and/or fermentation can be treated to remove solids, For example, by centrifugation, filtration, screening, or rotary vacuum filtration. For example, some methods and equipment that can be used during or after saccharification are disclosed in PCT Application No. PCT/US14/21584 filed Mar. 7, 2014 and U.S. application Ser. No. 13/932,814 filed on Jul. 1, 2013, the entire disclosures of which are incorporated herein by reference. In addition other separation techniques can be used on the liquids. For example, to remove ions and de-colorize. For example, chromatography, simulated moving bed chromatograph and electrodialysis can be used to purify any of the solutions and or suspensions described herein. Some of these methods are discussed in PCT Application No. PCT/US14/21638 filed on Mar. 7, 2014 and PCT Application No. PCT/US14/21815 filed Mar. 7, 2014, the entire disclosures of which are incorporated herein by reference. Solids that are removed during the processing can be utilized for energy co-generation. For example, as discussed in PCT Application No. PCT/US14/21634, filed Mar. 7, 2014, the entire disclosure of which is herein incorporated by reference.

Optionally the sugars released from biomass as describe in herein. For example, glucose, xylose, sucrose, maltose, lactose, mannose, galactose, arabinose, homodimers and heterodimers of these (e.g., cellobiose, sucrose), trimers, oligomers and mixtures of these, can be fermented to poly carboxylic acids (e.g., succinic acid). Optionally, the saccarification and fermentation can be done simultaneously. In some instances, the biomass can be processed (e.g., fermented) to an alcohol (e.g., ethanol and glycol) and the alcohol can then be fermented to a poly carboxylic acid.

Poly carboxylic acids that can be produced by the methods systems and equipment described herein include, for example, organic compounds with two or more carboxylate groups where independently each of the carboxylate groups can be in the protonated form (e.g., acid), unprotonated form (e.g., conjugate base) or a salt thereof. For example, the salt can be the salt of any positively charged ion. For example, ions of metals, or metal compounds such as hydroxides, derived from alkai metals such as Li, Na, K, Cs, alkali earth metals such as Mg, Ca, Sr Ba, transition metals such as Mn, Fe, Co, Ni, Cu, Zn, lanthanides such as La and Ce and main group elements such as B, Al and Ga. In some embodiments the poly carboxylic acid forms a coordination compound or covalently bonded compound to the metal ions rather than a salt.

The poly carboxylic acid can be represented, for example, in its protonated form by the formula:

Cₘ(CO₂H)ₙ(X)ₒH₂ₘ₋ₙ₋ₒ₊₂

Where m is at least 1 and n is an integer chosen from 2 through and including 2 m. “X” is any functional group or functional groups, for example, hydrogen, amine, alkyl, alkyne, arene, aromatic, benzyl, ketone, ether, ester, aldehyde, amide, alcohol, thiol, cyano, sulfate, phosphate, halide (e.g., chloride, bromide), a ring structure (such as heteroatom containing aromatic group e.g., pyridine), a protein, a metal (e.g., ions of metals, or metal compounds such as hydoxides derived from alkai metals such as Li, Na, K, Cs, alkali earth metals such as Mg, Ca, Sr Ba, transition metals such as Mn, Fe, Co, Ni, Cu, Zn, lanthanides such as La and Ce and main group elements such as B, aluminum and gallium), selections of one or more of these and combinations of these. The value of o is an integer chosen from 0 through and including 2 m. The poly carboxylic acid can include a mixture of compounds with different values of m, n and o. Preferably, m is an integer chosen from 1 through and including 20 and n is a number chosen from 2 through and including 4.

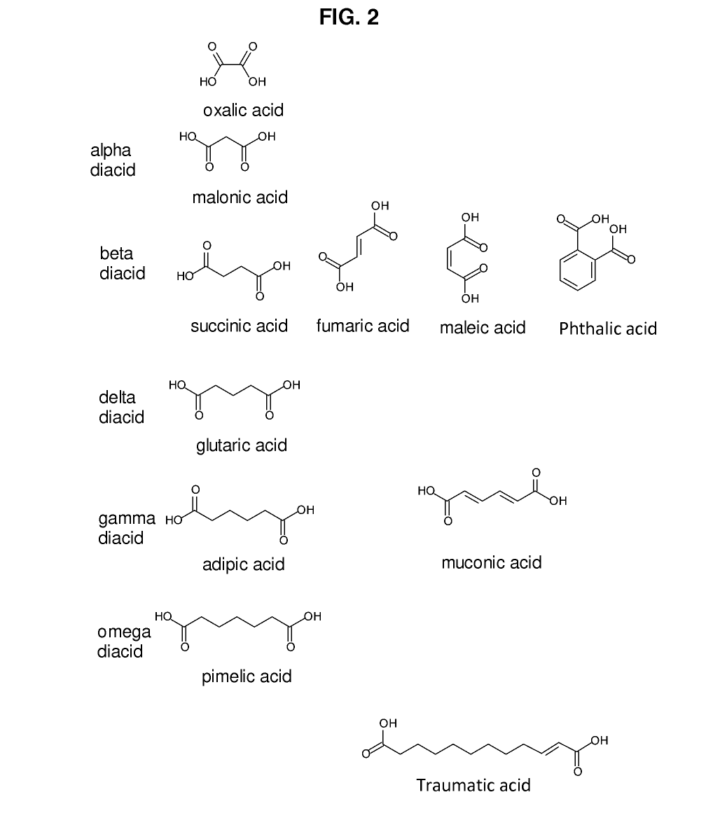

The structures of the poly carboxylic acids can include linear structures, branched structures, cyclic structures, fused cyclic structures and can have different substitution patterns (e.g., alpha, beta, delta, gamma, omega di-acids) and combinations of these structures. For example, some examples of poly carboxylic acids include oxalic acid, malonic acid, succinic acid tartaric, glutaric acid, adipic acid, pimelic acid, suberic acid, azelaic acid, sebacic acid, undecanedioic acid, dodecanedioic acid, maleic acid, fumaric acid, glutaconic acid, traumatic acid, muconic acid, phthalic acid, isophthalic acid, terephthalic acid, citric acid, isocitric acid, aconitic acid, mellitic acid and mixtures of these. Some of the structures are shown in FIG. 2.

### Preparation of Succinic Acid

Succinic acid can be extracted from natural sources, such as from amber, and is also known as Spirits of Amber. Succinic acid is also prevalent in biological systems playing a role in the Krebs cycle (e.g., also known as the citric acid and tricarboxylic acid cycle). Some organisms can, for example, utilize the reductive Krebs cycle to produce succinate from pyruvate or pyruvate phosphoenolpyruvate (e.g., anaerobically fixing CO₂). Other pathways include fermentative oxidation by organisms wherein the Krebs cycle and glyoxylate cycle are activated under aerobic conditions.

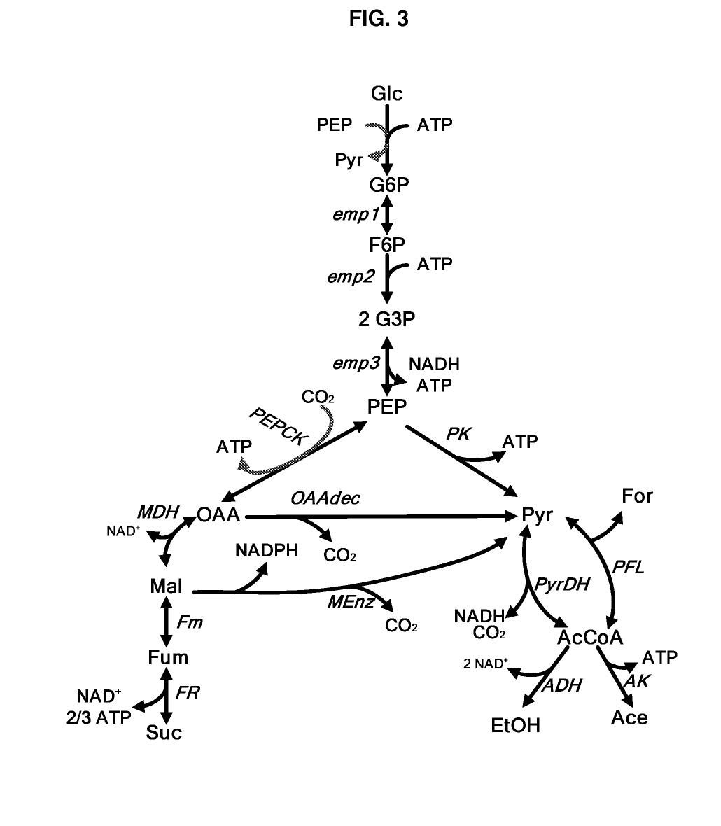

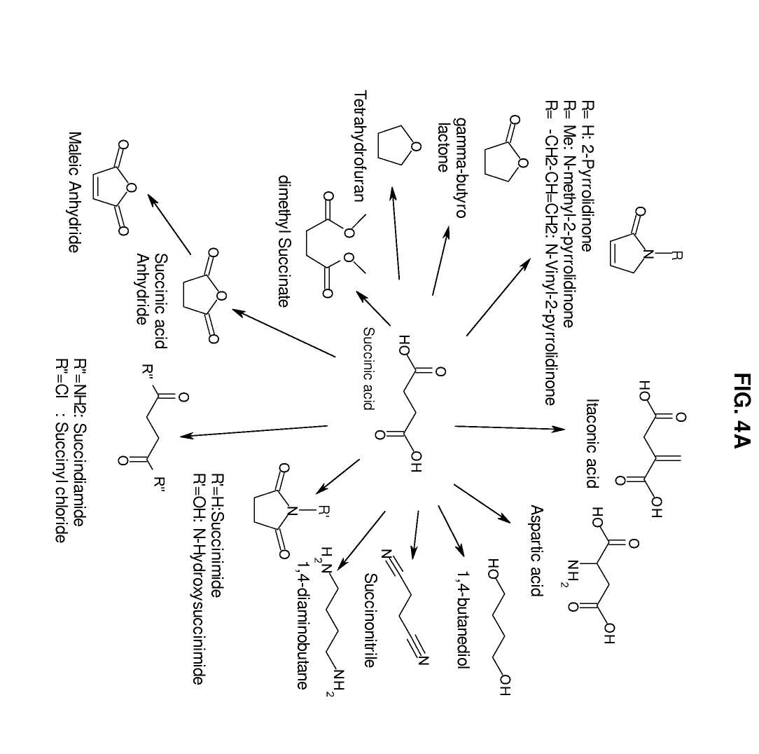

A possible biochemical pathway is shown in FIG. 3 for a fermenting organism to produce succinate. In a first stage, sugars such as glucose (Glc) are converted to phosphoenolpyruvate (PEP) through glycolysis steps, e.g., intermediates glucose 6-phosphate (G6P), fructose 6-phosphate (F6P) and glucose 3-phosphate (G3P). From PEP, the metabolic pathway can take one of two paths depending on the level of carbon dioxide available to the system. Under conditions of low carbon dioxide concentration, the preferred metabolic pathway shifts to the formation of pyruvate (Pyr) formate (For) and Acetyl-CoA (AcCoA) with ethanol (EtOH) and acetate (Ace) as typical end products. Under higher carbon dioxide concentration. For example, wherein the fermentation solution is sparged with carbon dioxide and is saturated or close to saturation, the microorganism favors the production oxaloacetate and then through Malate and Fumarate, succinic acid can be the final product. Sugars other than glucose, such as xylose, can be fermented using pentose phosphate pathways to also produce succinate.

Several organism, such as bacteria, yeasts and fungi, can be utilized to ferment biomass derived products such as sugars and alcohols to succinic acid. For example, organisms can be selected from; Actinobacillus succinogenes, Anaerobiospirillum succiniciproducens, Mannheimia succiniciproducens, Ruminococcus flaverfaciens, Ruminococcus albus, Fibrobacter succinogenes, Bacteroides fragilis, Bacteroides ruminicola, Bacteroides amylophilus, Bacteroides succinogenes, Mannheimia succiniciproducens, Corynebacterium glutamicum, Aspergillus niger, Aspergillus fumigatus, Byssochlamys nivea, Lentinus degener, Paecilomyces varioti, Penicillium viniferum, Saccharomyces cerevisiae, Enterococcus faecali, Prevotella ruminicolas, Debaryomyces hansenii, Candida catenulata VKM Y-5, C. mycoderma VKM Y-240, C. rugosa VKM Y-67, C. paludigena VKM Y-2443, C. utilis VKM Y-74, C. utilis 766, C. zeylanoides VKM Y-6, C. zeylanoides VKM Y-14, C. zeylanoides VKM Y-2324, C. zeylanoides VKM Y-1543, C. zeylanoides VKM Y-2595, C. valida VKM Y-934, Kluyveromyces wickerhamii VKM Y-589, Pichia anomala VKM Y-118, P. besseyi VKM Y-2084, P. media VKM Y-1381, P. guilliermondii H-P-4, P. guilliermondii 916, P. inositovora VKM Y-2494, Saccharomyces cerevisiae VKM Y-381, Torulopsis candida 127, T. candida 420, Yarrowia lipolytica 12a, Y. lipolytica VKM Y-47, Y. lipolytica 69, Y. lipolytica VKM Y-57, Y. lipolytica 212, Y. lipolytica 374/4, Y. lipolytica 585, Y. lipolytica 695, Y. lipolytica 704, and mixtures of these organisms.

In addition, genetically modified organisms can be utilized to produce poly carboxylic acids such as succinic acid (e.g., from biomass derived sugars and alcohols), For example, recombinant Escherichia coli, (e.g., PEP carboxylase over-expressing E. coli) and genetically modified Corynebacterium glutamicum can be utilized.

Co-cultures of organisms, for example, chosen from organisms as describe herein, can be used in the fermentations of biomass derived products (e.g., sugars and alcohols) to poly carboxylic acids in any combination. For example, two or more bacteria, yeasts and/or fungi can be combined with one or more sugar/alcohol (e.g., ethanol, glycol, glucose and/or xylose) where the organisms ferment the sugar/alcohol together, selectively and/or sequentially. Optionally, one organism can be added first and the fermentation proceed for a time. For example, until it stops fermenting one or more of the sugars, and then a second organism can be added to further ferment the same sugar or ferment a different sugar. For example, fumaric acid that is obtained from the fermentation of glucose using Rhizopus sp. can be subsequently converted to succinic acid by E. faecalis. Or a yeast that ferments glucose to ethanol, such as baker's yeast, can be combined with a yeast, such as Pichia anomal that ferments alcohols to succinic acid.

In some embodiments additives (e.g., media components) can be added during the fermentation (e.g., with the saccharified biomass or biomass derived alcohol). For example, additives that can be utilized include sugars such as glucose, xylose and alcohols such as ethanol and glycol. Other optional additives include, for example, yeast extract, rice bran, wheat bran, corn steep liquor, black strap molasses, casein hydrolyzate, vegetable extracts, corn steep solid, corn steep liquor, ram horn waste, peptides, peptone (e.g., bactopeptone, polypeptone, soy peptone), pharmamedia, flower (e.g., wheat flour, soybean flour, cottonseed flour), malt extract, beef extract, tryptone, Flour hydrolysate, corn hydrolysate and fungal hydrolysate. Metals/minerals can also optionally be added to the fermentation media, for example, K₂HPO₄; KH₂PO₄; Na₂HPO₄; NaH₂PO₄; (NH₄)₂PO₄; NaCl; MgCl₂.6H₂O; CaCl₂.2H₂O; MgCO₃; MnSO₄.5H₂O; MgSO₄.7H₂O; CaCl₂.2H₂O; FeSO₄.7H₂O; CoCl.6H₂O; Na₂MoO₄; NiCl₂.6H2O; Na₂WO₄.2H₂O; ZnCl₂; ZnSO₄; CuSO₄.5H₂O; AlK(SO₄)₂.12H₂O; H₃BO₃; NaSeO₃. Vitamins, such as thiamine, riboflavin, niacin, niacinamide, pantothenic acid, pyridoxine, pyridoxal, pyridoxamine, pyridoxine hydrochloride, biotin, folic acid, p-aminobenzoate, lipoic acid can also be added. Addition of protease can also be beneficial during the fermentation. Optionally, surfactants such as Tween 80 and antibiotics such as choloramphenicol can also be beneficial. Antifoaming compounds such as Antifoam 204 and/or AFE-0010 can also be utilized. In addition to these components, CO₂ can be added to the media. For example, using a gas sparging tube.

In some embodiments the fermentation can take from about 8 hours to several days. For example, some batch fermenations can take from about 1 to about 20 days (e.g., about 1-10 days, about 3-6 days, about 8 hours to 48 hours, about 8 hours to 24 hours).

In some embodiments the temperature during the fermentation is controlled. For example, the temperature can be controlled between about 20 deg C and 50 deg C (e.g., between about 25 and 40 deg C, between about 30 and 40 deg C, between about 35 and 40 deg. C). In some cases thermophilic organsims are utilized that operate efficiently above about 50 deg C, For example, between about 50 deg C and 100 deg. C (e.g., between about 50-90 deg. C, between about 50 to 80 deg. C, between about 50 to 70 deg. C).

In some embodiments the pH is controlled. For example, by the addition of an acid or a base. The pH can be optionally controlled to be close to neutral (e.g., between about 4-8, between about 5-7, between about 5-6). Acids, for example, can be protic acids such as sulfuric, phosphoric, nitric, hydrochloric and acetic acids. Bases, for example, can include metal hydroxides and carbonates (e.g., sodium and potassium hydroxide), ammonium hydroxide, calcium carbonate and magnesium carbonate. Phosphate and other buffers can also be utilized. In some preferred embodiment the pH is controlled by the addition of sodium hydroxide.

Fermentation methods include, for example, batch, fed batch, repeated batch or continuous reactors. Often batch methods can produce higher concentrations of lactic acids, while continuous methods can lead to higher productivities.

Fed batch methods can include adding media components and substrate (e.g., sugars from biomass) as they are depleted. Optionally products, intermediates, side products and/or waste products can be removed as they are produced. In addition solvent (e.g., water) can be added or removed to maintain the optimal amount for the fermentation.

Options include cell-recycling. For example, using a hollow fiber membrane to separate cells from media components and products after fermentation is complete. The cells can then be re-utilized in repeated batches. In other optional methods the cells can be supported. For example, as described in U.S. application Ser. No. 13/293,971, filed on Nov. 10, 2011 and U.S. Pat. No. 8,377,668, issued Feb. 19, 2013 the entire disclosures of which are herein incorporated by reference.

### Purificaton of Succinic Acid

For many uses, the protonated form of succinic acid is required (e.g., for further conversion to useful products). Several methods can be utilized for product recovery, concentration and acidification from the fermentation broth. For example, reactive extraction, ion exchange resins, electrodialysis, precipitation, nanofiltration and simulated moving bed chromatography (SMB).

Amine-based extraction is a method of reactive extraction that separates organic acids (e.g., poly carboxylic acids such as succinic acid) based on their pKA values as it removes undissociated acids. Advantageously, this separation method is possible in-situ at room temperature and atmospheric pressure with no pre-treatment. For example, reactive extraction with tri-alkyl amines (e.g., tri-n-octylamine) can be utilized to extract the poly organic acids as they are produced or after a fermentation is complete into a hydrophobic phase. The extracted poly carboxylic acid/amine adduct can subsequently be acidified to release the poly carboxylic acid which can then be. For example, precipitated, crystalized, distilled or reacted. The pH during the extraction with tri-alkyl amines must be kept low. The method will extract most organic acids in the fermentation broth, so if other acids are present, further purification can be required.

Optionally, ion-exchange resins can be utilized to purify poly carboxylic acids (e.g., succinic acid) from, for example, fermentation broth. Ion exchange technology involves using a resin that captures cations with an ionic resin. For example, a cationic resin can be used to remove the organic acids. Alternatively a highly acidic ion exchange resin followed by a weak basic exchange resin can remove cations, anions and other impurities, leaving behind a purified stream with low concentrations of nitrogenous impurities, protein impurities, lignin impurities and sulphates. Preferably, a purification step to remove cells and other solids from the liquid is used prior to using a resin packed column. Alternatively, the exchange resins can be used in batch mode.

Some examples of adsorbents that can be used (e.g., in a column or added to a batch to purify during or after fermentation) include strong and weak base polymers, molecular sieves, and macroreticular resins. For example, Dow XUS 40285 Weak base polymer, Dow XUS 40091 Weak base, Dow XUS 40323 Strong base, Dow XUS 40283 Strong base, Dow XUS 43432 Weak base, Dow XUS 40196 Strong base, Dow XUS 40189 Strong base Polymer, Amberlite® IRA-93 RH Weak base macroreticular, Amberlite®IRA®-35 Weak base macroreticular, Amberlite® XAD-4 Weak base polymer, Amberliet® XAD-7 Weak base polymer, Dowex® 1×2 Weak base. Marathon® WBA Strong base macroreticular, Dowex® MSA-1® Strong acid PVP, Dowex® MSA-2® Strong acid PVP, REILLEX™ 425 PVP, REILLEX™ HPQ Hydrophobic molecular sieve, REILLEX™ 402 Polymer, SILICALITE™ powder hydrophobic molecular sieve, SILICALITE™ pellet hydrophobic molecular sieve with binder, AG-3® Styrene, AG-®1 Styrene quaternary amine, Dowex® MWA-1 Tertiary amine macroreticular, Hytrel® 8206 and Hytrel® G3548L.

Methods for recovering the poly carboxylic acid from the adsorbents include treatment with hot water, acids, bases, solvents and combinations of these. These treatments can release the poly carboxylic acids and regenerate the adsorbents.

Another optional method for purification of poly carboxylic acids (e.g., succinic acid) is electrodialysis. In the fermentation broth, the dissociated succinic acid is ionic while other components, such as proteins, amino acids, lignin derived substrates and carbohydrates, are either often weakly ionic or non-ionic. Electrodialysis can target the dissociated form of the succinic acid and removes it while leaving behind the other compounds. Optionally, electrodialysis can be used while the fermentation is taking place to remove the succinic acid and the remaining fluid including cells can be recycled back to the fermenter.

Precipitation of poly carboxylic acid (e.g., succinic acid) is another optional purification method. For example, the fermentation broth can be centrifuged and or filtered (e.g., press-filtered, rotary vacuum drum filtered) and the broth can then be treated with calcium hydroxide or calcium carbonate. Calcium succinate precipitates out of solution and can be isolated (e.g., filtered and washed) from non-precipitating species. Calcium succinate can then be acidified, e.g., using sulfuric acid producing solid calcium sulfate which is removed (e.g., filtered) from the soluble succinic acid.

Another possible method for purification includes cross-flow filtration technologies such as nano-filtration. In this method, the nano-filter can retain poly carboxylic acids such as succinic acids while letting less highly charged and smaller molecules pass through the membranes.

Optionally, reactive distillation/extraction can also be used to purify poly carboxylic acids. For example, the esterification with methanol provides the methyl ester which can be distillated and/or extracted and then the ester can be hydrolyzed to the acid. Esterification to other esters can also be used to facilitate the separation. For example, reactions with alcohols to the ethyl, propyl, butyl, hexyl, octyl or even esters with more than eight carbons can be formed and then extracted in a solvent or distilled.

Other potentially useful methods for purification of poly carboxylic acids include SMB. For example, to separate the acids from other fermentation products such as residual sugars. Optionally, the acid groups can be modified, e.g., by esterification. As in other chromatographic techniques, it is preferable that solutions are treated with SMB, that is, most solids are removed, e.g., by filtration, prior to SMB.

More than one method as described herein can be utilized. For example, filtrations followed by extractions, followed by crystallizations and/or distillations can be utilized.

### Conversions of Succinic Acid

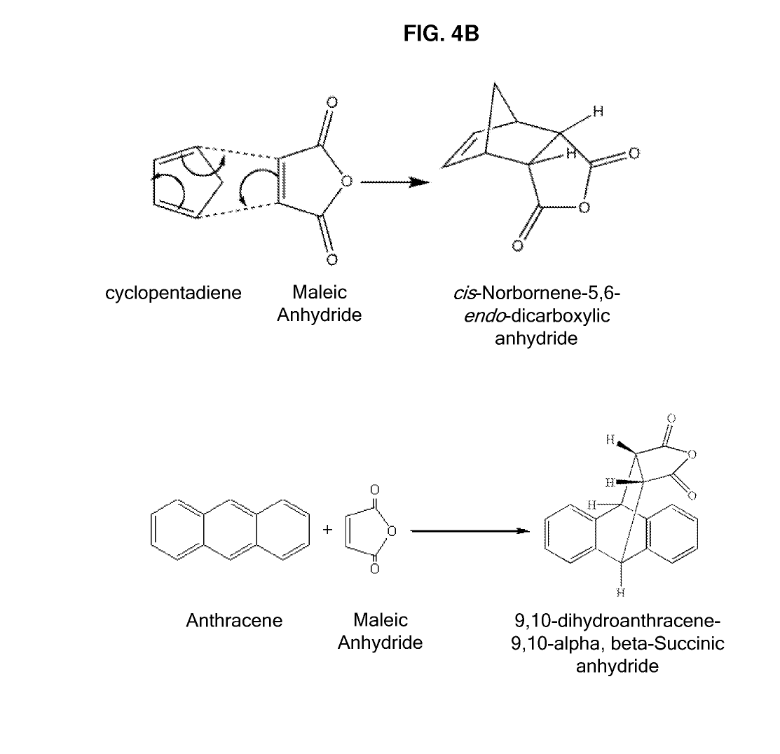

Succinic acid is a platform chemical to many important chemicals. For example, it is a substitute for petroleum chemicals such as butane and benzene which are themselves petrochemical routes to platform chemicals such as maleic anhydride. FIG. 4A shows some of the transformation (e.g., chemical transformations) available for succinic acid. Biological conversion is also possible.

Reactions including cyclizations and other reactions (e.g. amination, alkylations, oxidations, reductions, acid halide formation such as acid halide or acid bromide formation) can convert succinic acid to tetrahydrofuran, succinic acid anhydride, gamma-butyro lactone, 2-pyrrolidinone, N-methyl-2-pyrrolidinone (NMP), N-viny-2-pyrrolidinone, other N-substituted-2-pyrrolidinones (e.g., where R is an alkyl, aryl or other group), succinimide, succinyl chloride, N-hydroxysuccinimide and other N-substituted succinimides (e.g., where R is an alkyl, aryl, or other group). Compounds such as 2-pyrolidiones and succinimides have many applications/uses including, for example, as solvents, functionalizing agents for polymers (e.g., peptides, proteins and plastics), functional group activating agents, as intermediates to polymers such as polyvinylpyrrolidone and polypyrrolidone and as intermediates to pharmaceutical drugs such as cotinine, doxapram, piracetam, pvidone, phensuximide, methsuximide and ethosuximide. Tetrahydrofuran (THF) is an important solvent and precursor to poly-THF. Gamma-butyrolactone is a solvent, aroma compound, stain remover and chemical reagent.

Succinic acid is also a platform chemical to difunctional linear alkanes. For example, succindiamide, 1,4-diaminobutane, succinonitrile, 1,4-butandiol and dimethyl succinate. These chemicals have uses, for example, as intermediates to fine chemicals, solvents and as polymer precursors. For example; 1,4-diaminobutante (e.g., also known as putrescine) can be reacted with adipic acid to produce the polyaminde nylon-4,6; 1,4-butanediol is used in the manufacturing of plastics, elastomers, polyesters and polyurethanes; succinonitrile can used as a glazing agent in nickelizing, a battery solution additive, a raw material of quinacridone pigment and is intermediate an nylon-4.

Succinic acid anhydride can be dehydrogenated to maleic anhydride. Maleic anhydride can undergo many reactions including Diels Alder reactions. By way of example, FIG. 4B shows the reaction of succinic acid with cyclopentadiene producing the endo product, cis-Norbornene-5,6-endo-dicarboxylic anhydride, and the reaction of succinic acid with anthracene producing 9,10-dihydroanthracene-9,10-succinic anhydride. Other transformations of maleic acid can yield malic acid, tetrahydrophtalic anhydride, alpha olefin succinimides, succinyl chloride butanediol, tetrahydrofurane, polysuccinimides, ethyl vinyl acetate polymers, styrene copolymers, polyisobutenyl succinimides, unsaturated polyesters.

Other compounds that can be derived from succinic acid include tartaric acid, fumaric acid, aspartic acid and malic acid.

Polymers Made with Poly Carboxylic Acids

Some polymers can be made by the thermal polycondensation of polycarboxylic acids (e.g., dicarboxylic acids such as succinic acid and adipic acids) with diols, for example, 1,3-propanediol, 1,4-butane-diol, 1,5-pentanediol, 1,6-hexanediol, 1,4-cylcohexanedimethanol and mixtures of these can be used. This polycondensation often only provides a low molecular weight polymer. For example, with molecular weights of a few thousand. To increase the molecular weight chain extenders can be utilized. For example, a chain extension reagents include diepoxides (e.g., 1,3-butadiene diepoxide), di-acyl chloride (e.g., sebacoyl dichloride), diisocyanates (e.g., 4,4′-diphenylmethane, 2,4-toluene diisocyanate), phenols (e.g., bisphenol A), aromatic amines (e.g., 4,4′-diaminodiphenyl sulfone, 3,3′-dichloro-4,4′-diaminodiphenylmethane), phosgene (e.g., and phosgene substitutes such as diphosgene, triphosgene, carbonyl diimidazole, disuccinimidyl carbonate) and combinations of these. The use and choice of chain extenders can greatly modify the propertied of the polymer, for example, adding stiffness and thermal stability to the polymer. For example, reaction of the polyesters formed by the polycondensation of succinic acid with 1,3-propane diol, with chain extenders 4,4′-diisophenylmethane diisocyante and 1,3-propane diol produces a segmented polyester-polyurethane co polymer.

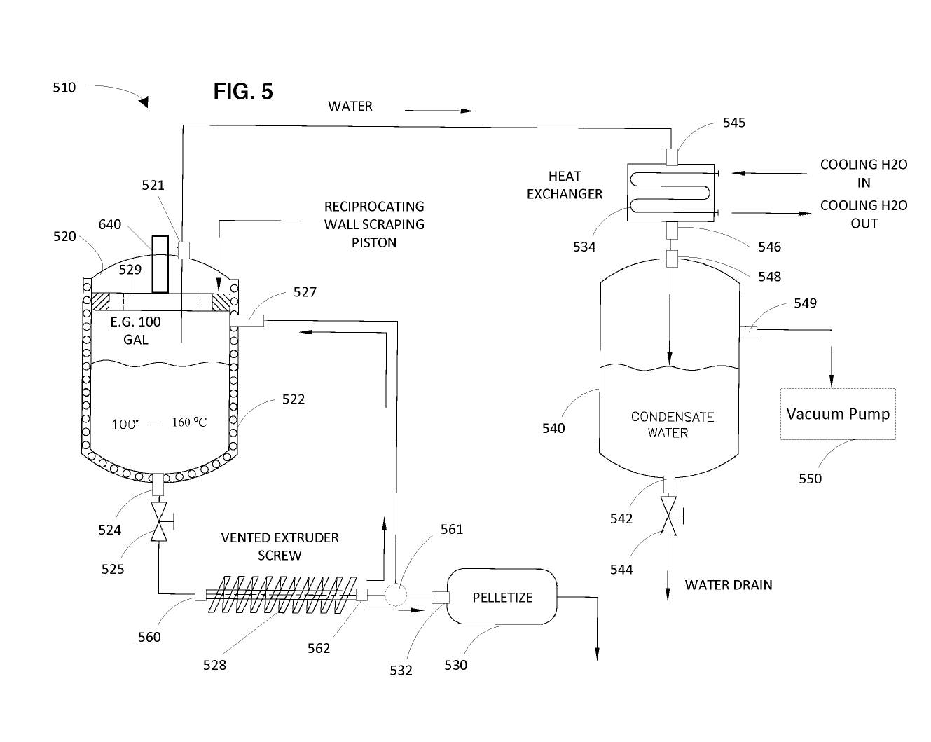

FIG. 5 shows a schematic view of a reaction system for polymerization of monomers for example, for making polyesters (e.g., including co-polymers of di acids and diols; poly lactic acids; and copolymers of D and/or L lactic acids, dicarboxylic acids and diamines) and polyurethanes). The reaction system 510 includes a stainless steel jacked reaction tank 520, a vented screw extruder 528, a pelletizer 530, a heat exchanger 534 and a condensation tank 540. An outlet 521 of the reaction tank is connected to a tube (e.g., stainless steel) which is connected to an inlet 545 to the heat exchanger. An outlet 546 to the heat exchanger is connected to another tube (e.g., stainless steel) and is connected to an inlet 548 to the condensation tank 540. The tubes and connections from the reaction tank and condensation tank provide a fluid pathway (e.g., water vapor/air) between the two tanks. A vacuum can be applied to the fluid pathway between the tanks 520 and 540 by utilizing a vacuum pump 550 that is connected to port 549.

The reaction tank 520 includes an outlet 524 that can be connected to a tube (e.g., stainless steel) that is connected to an inlet to a screw extruder 560. An outlet to the extruder 562 is connected to a tube which is connected optionally through a valve 561 to the reaction tank 520 through inlet 527. Optionally the outlet to the extruder 562 is connected through valve 561 to the pelletizer 530 through inlet 532. Tubes and connections from the reaction tank and extruder provide a circular fluid pathway (e.g., reactants and products) between the reaction tank and extruder when the valve 561 is set in recirculating position. The tubes and connections from the reaction tank to the pelletizer provide a fluid pathway between the reaction tank and pelletizer when the valve 561 is set in pelletizing position.

When in operation, the tank can be charged with monomers (e.g., diacids, diols D and/or L Lactic acid) or oligomers (e.g., low molecular weight polymers of monomers including diacids, diol, D and/or L Lactic acid). The monomers or oligomers can be heated in the tank utilizing the stainless steel heating jacket 522. In addition, a vacuum is applied to the condensation tank 540 and therefore to the reaction tank 520 through the stainless steel tubing and connections using the vacuum pump 550. The heating of the monomers (e.g., or oligomers) accelerates the condensation reactions (e.g., esterification reactions) to form oligomers (e.g., in cases where oligomers were added, to molecular weight of the oligomers can increase) while the applied vacuum helps volatilize the water that is produced. Water vapor travels out of the reactants and out of the reaction tank 520 and towards the heat exchanger 534 as indicated by the arrow. The heat exchanger cools the water vapor and the condensed water drops into the condensation tank 540 through the tubes and connections previously described. Multiple heat exchangers can be utilized.

In addition, during operation, extruder 528 can be engaged and operated to draw the reactants (e.g., monomers, oligomers and polymers) out of the tank. When the valve 561 is set in recirculating position the reactants/contents of tank 520 are circulated back to the reaction tank in the direction shown by the arrows. In addition to the extruder, the flow can be controlled by valve 525. For example, the valve can be set to closed for no flow, open for maximal flow or an intermediate position for lower or high flow rates (e.g., between about 0 and 100% open, e.g., about 0%, 10%, 20%, 30%, 40%, 50%, 60%, 70%, 80%, 90% or about 100% open).

The reaction can be continued with reactants following a circular pathway (e.g., with valve in recirculating position) until a desired polymerization degree, composition and/or polydispersity is achieved. This circulating pathway provides mixing and shearing that can help the polymerization (e.g., increase molecular weight, control polydispersity, improve the kinetics of the polymerization, improved temperature distribution and diffusion of reacting species). The products (e.g. polymer) can then be directed to the pelletizer by setting valve 561 to the pelletizing position. The pelletizer then can produce pellets which can be collected. Pellets can be of various shapes and sizes. For example, spherical or approximately spherical, hollow tube shaped, filled tube shape with, for example, approximate volumes, between about 1 mm³ to about 1 cm³. The pelletizer can also be replaced with other equipment. For example, extruders (e.g., film sheet or filament extruders), mixers, reactors, and filament makers.

The extruder 528 can be a vented screw extruder so that water or other volatile compounds can be removed from further processing. The extruder can be a single screw extruder or a multiple screw extruder. For example, the extruder can be a twine screw extruder with co-rotating or counter rotating screws. The screw extruder can also be a hollow flight extruder and can be heated or cooled. The screw extruder can be fitted with ports to its interior. The ports can be utilized, for example, for the addition of additives, addition of co-monomers, addition of cross-linking agents, addition of catalysts, addition of chain extending agents, irradiation treatments and addition of solvents. The ports can also be utilized for sampling (e.g., to test the progress of the reaction or troubleshoot the process). In addition to sampling, the torque applied to the extruder can be used to monitor the progress of the polymerization (e.g., as the viscosity increases). An in-line (e.g., a static mixer) mixer can also be disposed in the pathway of the circulating reactants, for example, before or after the screw extruder, providing a tortuous path for the reactants which can improve the mixing supplied to the reactants. The extruder can be sized, for example, so that the material is recirculated, e.g., about 0.25-10 times per hour (e.g., about 1-5 or 1-4 times per hour).

The position of the return port 527 allows the reactants to flow down the side of the tank, increasing the surface area of the reactants facilitating the removal of water. The return port can include multiple (e.g. a plurality of ports) disposed at various positions in the tanks. For example, the plurality of return ports can be placed circumferentially around the tank.

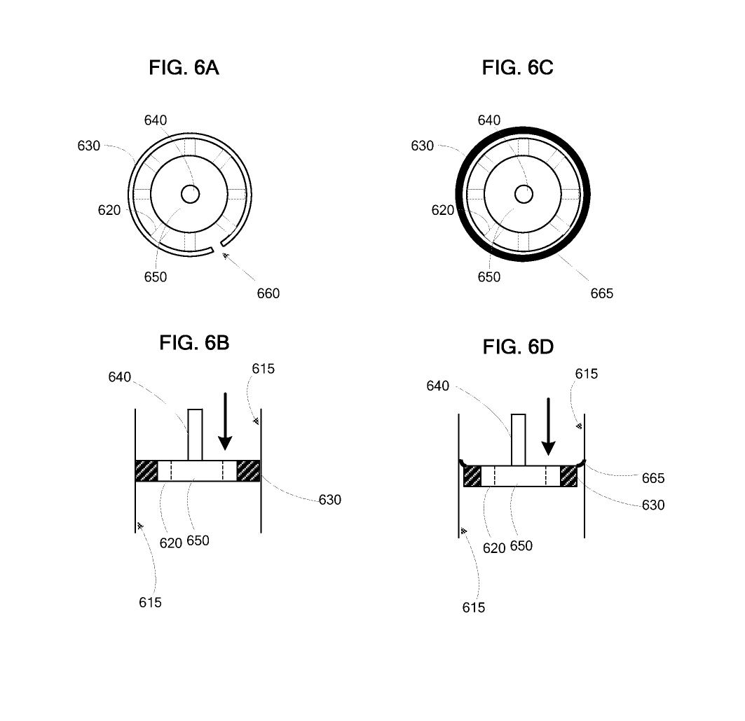

The tank can include a reciprocating scraper 529 which can help push the formed polymer/oligomers down the reaction tank. For example, during or after completion of the reaction. Once the reciprocating scraper moves down, the scraper can then be moved back up. For example, to a resting position. The scraper can be moved up and down the tank by engaging with an axel 640 that is attached to a hub 650 (seen in FIG. 6). In another possible embodiment, the hub can be tapped for mechanical coupling to a screw. For example, wherein the axel is a screw-axel that extends to the bottom of the tank. The screw-axel can then turn to drive the scraper down or up.

A top view of one embodiment of a reciprocating scraper is shown in FIG. 6A while a front cut out view is shown in FIG. 6B. The reciprocating scraper includes pistons 620 attached to a hub 650 and scraping ends 630. The scraping end is in the form of a compression ring with a gap 660. The pistons apply pressure against the inside surfaces of the tank 615 through the scraping ends 630 while the scraper can be moved down the tank as shown by the arrow in FIG. 6B. The gap 660 allows the expansion and contraction of the scraper. The scraper can be made of any flexible material. For example, steel such as stainless steel. The gap is preferably as small as possible (e.g., less than about 1″, less than about 0.1″, less than about 0.01″ or even less than about 0.001″).

Another embodiment of a reciprocating scraper is shown in FIG. 6C and FIG. 6D. In this second embodiment the scraping ends include a lip-seal. The lip seal can be made of a flexible material, for example, rubber. The movement of the lip-seal as the scraper moves up and down acts as a squeegee against the inside of the reaction tank.

The tank 520 can be 100 gal in size, although larger and smaller sizes can be utilized (e.g., between about 20 to 10,000 gal, e.g., at least 50 gal, at least 200 gal, at least 500 gal, at least 1000 gal). The tank, for example, can be shaped with a conical bottom or rounded bottom.

In addition to the inlets and outlets discussed, the tank can also include other openings. For example, to allow the addition of reagents or for access to the interior of the tank for repairs.

During the reaction the temperature in the tank can be controlled from between about 100 and 180 deg C. The polymerization can preferably started at about 100 deg C and the temperature increased to about 160 deg C over several hours (e.g., between 1 and 48 hours, 1 and 24 hours, 1 and 16 hours, 1 and 8 hours). A vacuum can be applied between about 0.1 and 2 mmHg). For example, at the beginning of the reaction about 0.1 mmHg and at the end of the reaction about 2 mmHg.

Water from the condenser tank 540 can be drained trough an opening 542 utilizing control valve 544.

The heat exchanger can be a fluid cooled heat exchanger. For example, cooled with water, air or oil. Several heat exchangers can be used, for example, as needed to condense as much of the water as possible. For example, a second heat exchanger can be located between the vacuum pump 550 and the condensation tank 540.

In some optional embodiments a monomer or monomer mixture is first dehydrated. For example, a monomer or monomer mixture can include a polycarboxylic acid. Polymerization can be considered as three steps or phases. The dehydrated mixture is oligomerized in a first step to a degree of polymerization of about 5 to about 50. In a second step the oligomers from the first step are heated to a temperature for melt polymerization, to a polymerization degree of between about 35 and to about 500. In a third step the polymer from step 2 is further polymerized. For example, the tank with the reciprocating scraper can be utilized, in any of the polymerization steps described.

In some optional embodiments a monomer or monomer mixture is first dehydrated. For example, a monomer or monomer mixture can include a polycarboxylic acid. Polymerization can be considered as three steps or phases. The dehydrated mixture is oligomerized in a first step to a degree of polymerization of about 5 to about 50. In a second step the oligomers from the first step are heated to a temperature for melt polymerization, to a polymerization degree of between about 35 and to about 500. In a third step the polymer from step 2 is further polymerized, for example, utilizing a devolitalizing device such as previously described (e.g., see FIGS. 5, 7a, 7b, 8a and 8b)

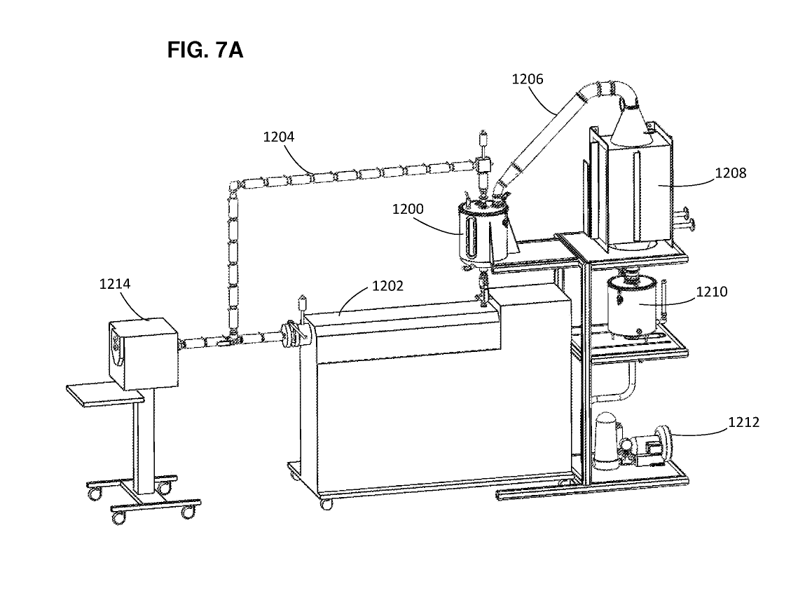

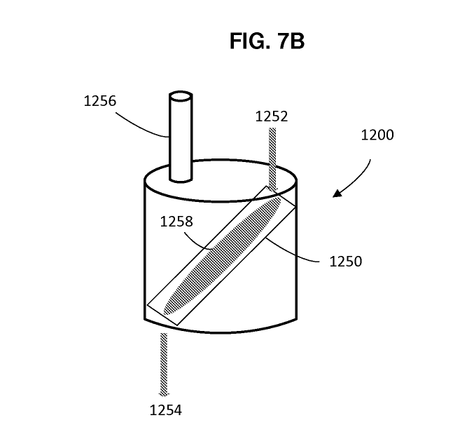

FIG. 7A is a schematic of a polymerization system for polymerizing or co-polymerizing e.g., hydroxyl carboxylic acid and/or polycarboxylic acids. The thin film evaporator or thin film polymerization/devolatilization device 1200, and (optional) extruder 1202 for product isolation or recycle back to the thin film evaporator or thin film polymerization/devolatilization device, a heated recycle loop 1204, a heated condenser 1206, cooled condenser 1208 for condensing water and other volatile components, a collection vessel 1210 a fluid transfer unit 1212 (e.g., including a pump) to remove condensed water and volatile components and a product isolation device 1214. The effluent from 1212 can optionally be taken to a another unit operation to recover the useful volatile components for recycle back to polymerization steps. For example, the first step discussed above. The thin film evaporator or thin film polymerization/devolatilization device is preferably utilized in the third step describe above. The fluid transfer unit is shown as a pump.

FIG. 7B is a cutaway of the thin film polymerization/devolatilization device. The angled rectangular piece 1250 is the optionally heated surface where the molten polymer flows. The incoming molten polymer stream 1252 flows onto the surface and is shown as an ellipse 1258 of flowing polymer flowing to the exit of the device at 1254. The volatiles are removed through pipe 1256.

The internals of the thin film evaporator or thin film polymerization/devolatilization device can be in different configurations, but can be configured to assure that the polymer fluid flows in a thin film through the device. This is to facilitate volatilization of the water that is in the polymer fluid or is formed by a condensation reaction. For instance, the surface may be slanted at an angle relative to the straight sides of the device. The surface may be separately heated such that the surface is 0 to 40° C. hotter than the polymer fluid. With this heated surface it can be heated to up to 300° C., as much as 40° C. higher than the overall temperature of the device.

The thickness of the polymer fluid flowing along the thin film part of the device is less than 1 cm, optionally less than 0.5 cm or alternately less than 0.25 cm.

The thin film evaporator and thin film polymerization/devolatilization device are similar in function. Other similar devices similar in function should be considered to have the same function as these. Descriptively, these include wiped film evaporators (e.g., as previously described), short path evaporator, a shell and tube heat exchanger and the like. For each of these evaporator configurations a distributor may be used to assure distribution of the thin film. The limitation that they must be able to operate at the conditions described above.

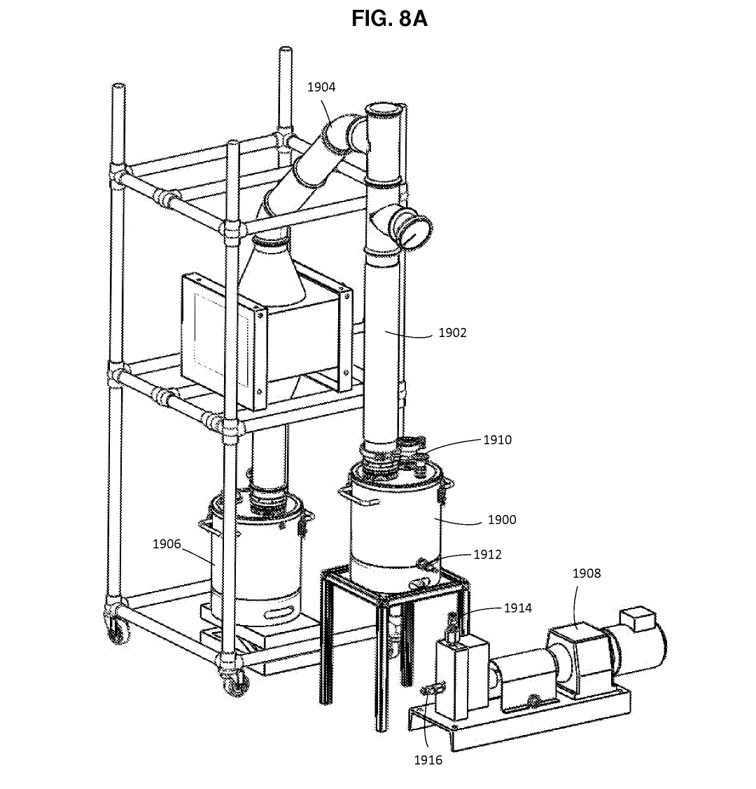

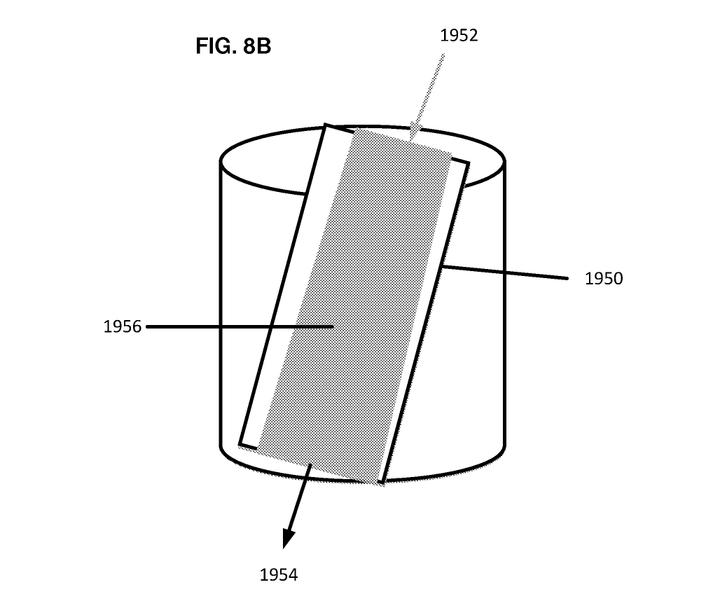

FIG. 8A is a schematic of a pilot-scale polymerization system to polymerize hydroxy carboxylic acid and/or poly carboxylic acid. The thin film evaporator or thin film polymerization/devolatilization device 1900, a heated riser 1902, a cooled condenser 1904 for condensing water and other volatile components, a collection vessel 1906 a fluid transfer unit 1908 to recycle the polymer fluid shown as a pump. The connecting tubing is not shown for clarity. The output of the pump 1916 is connected to inlet 1910, the device output 1912 is connected to the inlet of the pump 1914. The product isolation section is not shown. Internal in the thin film polymerization/devolatilization device is a slanted surface. The polymer fluid is flowed to the inlet with the configured such that the polymer fluid flows onto the slanted surface. This slanted surface may be separately heated as described above.

FIG. 8B is a cutaway of the thin film polymerization/devolatilization device. The angled rectangular piece 1950 is the optionally heated surface where the molten polymer flows. The incoming molten polymer stream 1952 flows onto the surface and is shown as a trapezoid 1956 of flowing polymer flowing to the exit of the device at 1954.

The polymerization systems and devices described can be made of any normally used metals for chemical processing equipment. Since the carboxylic acids and poly carboxylic acids can be corrosive the thin film evaporator may be clad or coated with corrosive resistant metals such as tantalum, alloys such as Hastelloy™, a trademarked alloy from Haynes International, and the like. It can also be coated with inert high temperature polymeric coatings such as Teflon™ from DuPont, Wilmington Del. For example, the corrosivity of the hydroxy-carboxylic acid system may not be surprising since the pKa of lactic acid is more than 0.8 less than acetic acid. Also, water undoubtedly hydrates the acid and the acid end of the polymer. When those waters of hydration are removed the acidity can be much higher, since it is not leveled by the waters of hydration.

Optionally, polymerizations can be done utilizing catalysts and/or promoters. The catalyst can be added after a desired degree of polymerization is obtained. For example, protonic acids and Lewis acids may be used. Examples of the acids include sulfonic acids, H₃PO₄, H₂SO₄, sulfonic acids, e, g, methane sulfonic acid, p-toluene sulfonic acid, Nafion® NR 50H+ form From DuPont, Wilmington Del. (sulfonic acid supported/bonded to a polymer that optionally may have a tetrafluorethylene backbone), acids supported on or bonded onto polymers, metals, Mg, Al, Ti, Zn, Sn, metal oxides, TiO₂, ZnO, GeO₂, ZrO₂, SnO, SnO₂, Sb₂O₃, metal halides, ZnCl₂, SnCl₂, AlCl₃ SnCl₄, Mn(AcO)₂, Fe₂(LA)₃, Co(AcO)₂, Ni(AcO)₂, Cu(OAc)₂, Zn(LA)₂, Y(OAc)₃, Al(i-PrO)₃, Ti(BuO)₄, TiO(acac)₂, (Bu)₂SnO, tin octoate, solvates of any of these and mixtures of these can be used. For instance, p-toluene sulfonic acid and tin octoate or tin chloride may be used together.

In some embodiments, the polymerizations can be done at a temperature between about 100 and about 260° C., such as between about 110 and about 240° C. or between about 120 and about 200° C. Optionally, at least a portion of the polymerizations can be performed under vacuum (e.g., between about 0.005 to 300 kPa).

After the polymerization has reached the desired molecular weight, it may be necessary to deactivate and/or remove the catalyst from the polymer. The catalyst can be reacted with a variety of compounds, including, silica, functionalized silica, alumina, clays, functionalized clays, amines, carboxylic acids, phosphites, acetic anhydride, functionalized polymers, EDTA and similar chelating agents.

While not being bound by theory for those catalysts like the tin systems, if the added compound can occupy multiple sites on the tin it can be rendered inactive for polymerization (and depolymerization). For example, a compound like EDTA can occupy several sites in the coordination sphere of the tin and, in turn, interfere with the catalytic sites in the coordination sphere. Alternatively, the added compound can be of sufficient size and the catalyst can adhere to its surface, such that the absorbed catalyst may be filtered from the polymer. Those added compounds such as silica may have sufficient acidic/basic properties that the silica adsorbs the catalyst and is filterable.

Optionally, the catalyst may be removed from the molten polymer. Removing catalyst may be accomplished just prior to, during, or after utilizing the polymerization device. The catalyst may be filtered from the molten polymer by using a filtration system similar to a screen pack. For example, since the molten polymer is flowing around the thin film evaporator/thin film polymerization/devolatilization device, a filtration system can be added. Alternatively, since the polymer flows through a screw extruder (e.g., with respect to FIG. 5) a filtering system can be added in line with the screw extruder.

To facilitate the catalyst removal, a neutralization or chelation chemical may be added. Candidate compounds include phosphites, anhydrides, poly carboxylic acids, polyamines, hydrazides, EDTA (and similar compounds) and the like. These neutralization and/or chelation compounds can be insoluble in the molten polymer leading to facile filtration. Poly carboxylic acids include poly acrylic acids and poly methacrylic acids. The latter can be in a both a random, block, and graft polymer configuration. The amines include ethylene diamine, oligomers of ethylene diamine and other similar polyamines such as methyl bis-3-amino, propane.

Another option to remove the catalyst includes adding solid materials to the polymer melt. Examples of added materials include silica, alumina, aluminosilicates, clays, diatomaceous earth, polymers and like solid materials. Each of these can be optionally functionalized to react/bind with the catalyst. When the catalyst binds/bonds to these structures it can be filtered from the polymer.

The (co)polymer product is isolated when the desired conversion/physical properties are achieved. The product can be conveyed to a product collection/isolation area. Optionally, a final devolatilization step may be performed just prior to product isolation. Types of equipment to isolate the (co)polymer product can include rotoform pastillation system and similar systems in which the product is cooled to obtain a product in a useable form. Optionally, the final product can be directed to a pelletizer as previously discussed, to form pellets.

The equipment and reactions described herein (e.g., as described with reference to FIGS. 5, 7a, 7b, 8a and 8b) can also be used for polymerization of other monomers. In addition, the equipment can be utilized after or during the polymerizations for blending of polymers. For example, any of the hydroxyl acids and poly carboxylic acids described herein can be polymerized by the methods, equipment and system described herein.

In addition to chemical method, lactic acid can be polymerized by LA-polymerizing enzymes and organisms. For example, ring opening polymerization (ROP) can be catalyzed by Candida antarctica lipase B, and hydrolases.

Surfactants Made with Poly Carboxylic Acids

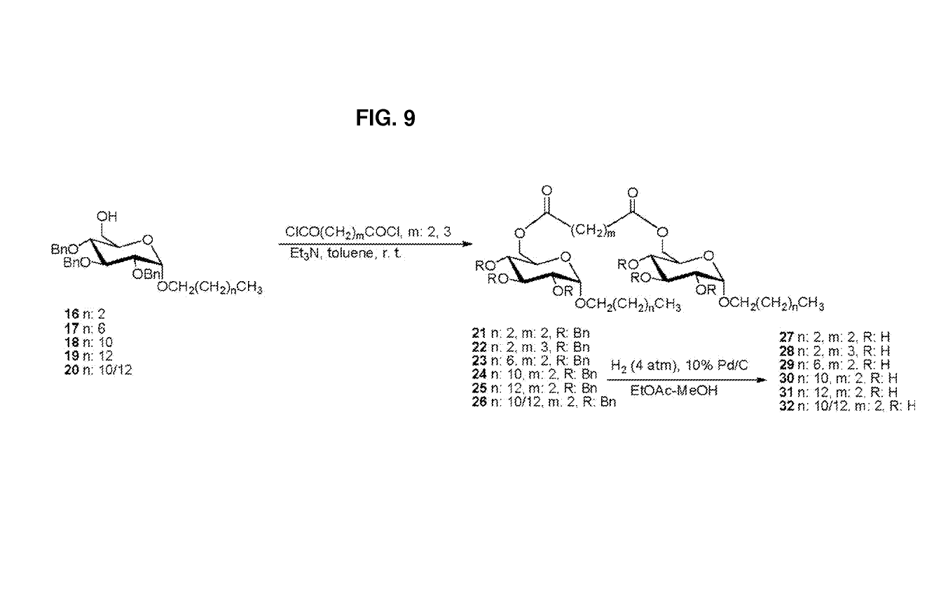

Poly carboxylic acids can be used to prepare surfactants. For example, dicarboxylic acids such as succinic, glutaric, terpthalic and adipic acid or any other diacid such as those describe herein can be utilized to prepare surfactants by condensation of one of the acid groups with a, for example, a long chain alcohol or a Gemini surfactants can be made by reacting both acid groups. For example, alcohols with between about 10 and about 40 carbon atoms (e.g., or even longer chains such as at least 40, at least 50, at least 60) such as Cetostearyl alcohol, geddyl alcohol, 1-dotriacontanol, myricyl alcohol, 1-nonacosanol, montanyl alcohol, 1-heptacosanol, ceryl alcohol, lignoceryl alcohol, erucyl alcohol, behenyl alcohol, heneicosyl alcohol, arachidyl alcohol, nonadecyl alcohol, stearyl alcohol, heptadecyl alcohol, palmitoleyl alcohol, cetyl alcohol, pentadecyl alcohol, myristyl alcohol, tridecyl alcohol, lauryl alcohol, undecyl alcohol, capric alcohol. Optionally, other groups can be used to form a link to the di-acid. For example, surfactants can be produced as described in Mariano et al. ARKIVOC 2005 (xii) 253-267 shown in FIG. 9. In this preparation, butyl (n=2), octyl (n=6), dodecyl (n=10), tetradecyl (n=12) and dodecyl/tetradecyl (n=10/12) alpha-glucopyranosides are first prepared by Fisher glycosidation and acetylation of D-glucose. Compounds 16-20 are then prepared by the benylzylation of carbons 1 through 4. The compounds are condensed with the acid chloride of succinic acid (m=2) or glutaric acid (m=3) producing compounds 21-26. The hydrogenation of 21-26 yields glucoside-based gemini surfactants 27-32. The glucose can be prepared from biomass material. For example, as described herein. Other sugars, such as xylose, can be used to make surfactants in a similar manner. Other acyl groups can be used to make the alpha-glucopyranosides such as with more than 14 carbon chains (e.g., from 14 to 40 carbon chains or more).

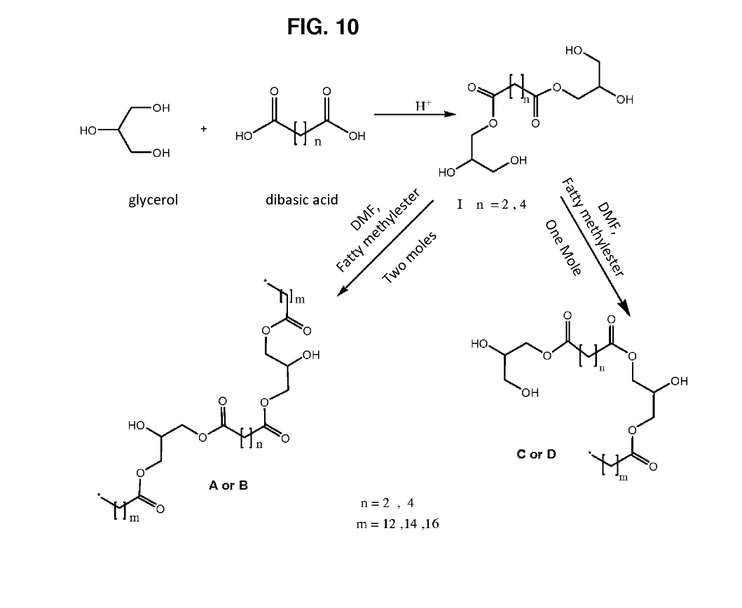

Di-acids can also be reacted with alcohols with more than 2 hydroxyl groups, for example, such as glycerol and then further reacted, for example, with saturated and unsaturated fatty acids (e.g., with between about 10 and 30 carbon atoms) on one or both ends to form non-ionic surfactants. An example of the preparation of these kinds of surfactants is shown in FIG. 10, as described in Kandeel, Der chemical Sinica, 2011, 2(3):88-98. The compound A with n=2 is a 3-acyloxy-2-hydroxypropyl 2,3-dihydroxypropyl succinate; B with n=4 is 3-acyloxy-2-hydroxypropyl 2,3-dihyroxypropyl adipate; C with n=4 is bis(3-acyloxy-2-hydroxypropyl)succinate and; D with n=4 is bis(3-acyloxy-2-hydroxypropyl)adipate. The acyloxy groups are derived from lauric acid (m=12), myristic acid (m=14) and Palmitic acid (m=16).

Succinic acid can also be made into sulfosuccinates surfactants. A general structure is shown here as Structure I:

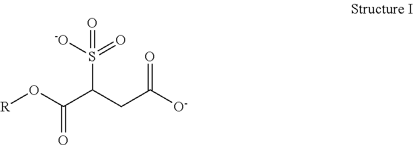

With regards to Structure I, counter ions (not shown) can be any positive ion. For example, ions of, alkai metals (e.g., Li, Na, K, Cs), alkai earth metals (Mg, Ca, Sr, Ba), transition metals and lanthnaides. Preferably Na¹⁺, K¹⁺, Mg²⁺ and Ca²⁺ are used as counter ions. The R group can be, for example, a long chain saturated, unsaturated, branched or unbranched alkyl group, a poly ether (e.g., poly ethylene oxide, poly propylene oxide), silicones or combinations of these groups. The chains can also include other groups such as aromatic groups, esters, ketones, amines, alcohols, cyclic aliphatic groups and combinations of these groups. For example, some sulfosuccinates that can be prepared from succinic acid and succinic acid derivatives are Disodium Laureth Sulfosuccinate; Disodium Laureth-6 Sulfosuccinate; Disodium Laureth-9 Sulfosuccinate; Disodium Laureth-12 sulfosuccinate; Disodium Deceth-5 Sulfosuccinate; Disodium Deceth-6 sulfosuccinate; Magnesium Laureth-3 Sulfosuccinate; Disodium C12-14 Pareth-1 Sulfosuccinate; Disodium C12-14 Pareth-2 Sulfosuccinate; Disodium C12-15 Pareth Sulfosuccinate; Disodium C12-14 Sec-Pareth-3 Sulfosuccinate; Disodium C12-14 Sec-Pareth-5 Sulfosuccinate; Disodium C12-14 Sec-Pareth-7 Sulfosuccinate; Disodium C12-14 Sec-Pareth-9 Sulfosuccinate; Disodium C12-14 Sec-Pareth-12 Sulfosuccinate; Disodium Oleth-3 Sulfosuccinate; Trisodium Sulfosuccinate; Disodium Laneth-5 Sulfosuccinate and; Disodium Coceth-3 Sulfosuccinate.

### Other Uses for Succinic Acid and Succinic Acid Derivatives

Succinic acid and derivatives of succinic acid can be used for the production of biopolymers such as polyesters (e.g., polyaspartic acid, polysuccinimides), polyamides, polyurethanes, polyols, poly-butylene succinate, styrene copolymers, polyisobutenyl succinimides, EVA copolymers, copolymers and blends of these polymers.

As well as being sourced from renewable materials the polymers can be composted, recycled, used as a fuel (incinerated). Some of the degradation reactions include thermal degradation, hydrolytic degradation and biotic degradations.

End product markets include personal care items, green packaging, gardening (e.g., pots), consumer electronics, appliances, food packaging, disposable packaging, garbage bags, mulch films, controlled release matrices and containers (e.g., for fertilizers, pesticides, herbicides, nutrients, pharmaceuticals, flavoring agents, foods), shopping bags, general purpose film, high heat film, heat seal layer, surface coating, disposable tableware (e.g., plates, cups, forks, knives, spoons, sporks, bowls), automotive parts (e.g., panels, fabrics, under hood covers), carpet fibers, clothing fibers and thread (e.g., for garments, sportswear, footwear), biomedical applications and engineering plastics.

In addition to polymers, succinic acid, succinic acid derivatives and similar compounds (e.g., poly carboxylic acids) coolants, deicers (e.g., succinate salts), cosmetics, personal care products, food, pharmaceuticals, agrichemicals, chemical intermediates, fine chemicals, solvents, plasticizers (e.g., succinate esters), fuel additives (e.g., succinate esters), corrosion inhibitors, plating compounds, detergents, surfactants, foaming agents, lubricant additives and chelators (e.g., for metals).

### Radiation Treatment

The feedstock can be treated with radiation to modify its structure to reduce its recalcitrance. Such treatment can, for example, reduce the average molecular weight of the feedstock, change the crystalline structure of the feedstock, and/or increase the surface area and/or porosity of the feedstock. Radiation can be by, for example, electron beam, ion beam, 100 nm to 28 nm ultraviolet (UV) light, gamma or X-ray radiation. Radiation treatments and systems for treatments are discussed in U.S. Pat. No. 8,142,620 and U.S. Publication No. US 2010-009324, published on Apr. 15, 2010, the entire disclosures of which are incorporated herein by reference.

Each form of radiation ionizes the biomass via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. Electrons interact via Coulomb scattering and bremsstrahlung radiation produced by changes in the velocity of electrons.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired to change the molecular structure of the carbohydrate containing material, positively charged particles may be desirable, in part, due to their acidic nature. When particles are utilized, the particles can have the mass of a resting electron, or greater, e.g., 500, 1000, 1500, or 2000 or more times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 atomic units.

Gamma radiation has the advantage of a significant penetration depth into a variety of material in the sample.

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ Hz, greater than 10¹⁷ Hz, 10¹⁸, 10¹⁹, 10²⁰, or even greater than 10²¹ Hz. In some embodiments, the electromagnetic radiation has a frequency of between 10¹⁸ and 10²² Hz, e.g., between 10¹⁹ to 10²¹ Hz.

Electron bombardment may be performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV.

The electron beam may have a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150 kW. In some cases, the power is even as high as 500 kW, 750 kW, or even 1000 kW or more. In some cases the electron beam has a beam power of 1200 kW or more, e.g., 1400, 1600, 1800, or even 3000 kW.

This high total beam power is usually achieved by utilizing multiple accelerating heads. For example, the electron beam device may include two, four, or more accelerating heads. The use of multiple heads, each of which has a relatively low beam power, prevents excessive temperature rise in the material, thereby preventing burning of the material, and also increases the uniformity of the dose through the thickness of the layer of material.

It is generally preferred that the bed of biomass material has a relatively uniform thickness. In some embodiments the thickness is less than about 1 inch (e.g., less than about 0.75 inches, less than about 0.5 inches, less than about 0.25 inches, less than about 0.1 inches, between about 0.1 and 1 inch, between about 0.2 and 0.3 inches).

It is desirable to treat the material as quickly as possible. In general, it is preferred that treatment be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 2 Mrad per second. Higher dose rates allow a higher throughput for a target (e.g., the desired) dose. Higher dose rates generally require higher line speeds, to avoid thermal decomposition of the material. In one implementation, the accelerator is set for 3 MeV, 50 mA beam current, and the line speed is 24 feet/minute, for a sample thickness of about 20 mm (e g, comminuted corn cob material with a bulk density of 0.5 g/cm³).

In some embodiments, electron bombardment is performed until the material receives a total dose of at least 0.1 Mrad, 0.25 Mrad, 1 Mrad, 5 Mrad, e.g., at least 10, 20, 30 or at least 40 Mrad. In some embodiments, the treatment is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of passes, e.g., at 5 Mrad/pass with each pass being applied for about one second. Cooling methods, systems and equipment can be used before, during, after and in between radiations, for example, utilizing a cooling screw conveyor and/or a cooled vibratory conveyor.

Using multiple heads as discussed above, the material can be treated in multiple passes, for example, two passes at 10 to 20 Mrad/pass, e.g., 12 to 18 Mrad/pass, separated by a few seconds of cool-down, or three passes of 7 to 12 Mrad/pass, e.g., 5 to 20 Mrad/pass, 10 to 40 Mrad/pass, 9 to 11 Mrad/pass. As discussed herein, treating the material with several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance treatment uniformity.

In some embodiments, electrons are accelerated to, for example, a speed of greater than 75 percent of the speed of light, e.g., greater than 85, 90, 95, or 99 percent of the speed of light.

In some embodiments, any processing described herein occurs on lignocellulosic material that remains dry as acquired or that has been dried, e.g., using heat and/or reduced pressure. For example, in some embodiments, the cellulosic and/or lignocellulosic material has less than about 25 wt. % retained water, measured at 25° C. and at fifty percent relative humidity (e.g., less than about 20 wt. %, less than about 15 wt. %, less than about 14 wt. %, less than about 13 wt. %, less than about 12 wt. %, less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, or less than about 0.5 wt. %.

In some embodiments, two or more ionizing sources can be used, such as two or more electron sources. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light. The biomass is conveyed through the treatment zone where it can be bombarded with electrons.

It may be advantageous to repeat the treatment to more thoroughly reduce the recalcitrance of the biomass and/or further modify the biomass. In particular the process parameters can be adjusted after a first (e.g., second, third, fourth or more) pass depending on the recalcitrance of the material. In some embodiments, a conveyor can be used which includes a circular system where the biomass is conveyed multiple times through the various processes described above. In some other embodiments, multiple treatment devices (e.g., electron beam generators) are used to treat the biomass multiple (e.g., 2, 3, 4 or more) times. In yet other embodiments, a single electron beam generator may be the source of multiple beams (e.g., 2, 3, 4 or more beams) that can be used for treatment of the biomass.

The effectiveness in changing the molecular/supermolecular structure and/or reducing the recalcitrance of the carbohydrate-containing biomass depends on the electron energy used and the dose applied, while exposure time depends on the power and dose. In some embodiments, the dose rate and total dose are adjusted so as not to destroy (e.g., char or burn) the biomass material. For example, the carbohydrates should not be damaged in the processing so that they can be released from the biomass intact, e.g. as monomeric sugars.

In some embodiments, the treatment (with any electron source or a combination of sources) is performed until the material receives a dose of at least about 0.05 Mrad, e.g., at least about 0.1, 0.25, 0.5, 0.75, 1.0, 2.5, 5.0, 7.5, 10.0, 15, 20, 25, 30, 40, 50, 60, 70, 80, 90, 100, 125, 150, 175, or 200 Mrad. In some embodiments, the treatment is performed until the material receives a dose of between 0.1-100 Mrad, 1-200, 5-200, 10-200, 5-150, 50-150 Mrad, 5-100, 5-50, 5-40, 10-50, 10-75, 15-50, 20-35 Mrad.

In some embodiments, relatively low doses of radiation are utilized, e.g., to increase the molecular weight of a cellulosic or lignocellulosic material (with any radiation source or a combination of sources described herein). For example, a dose of at least about 0.05 Mrad, e.g., at least about 0.1 Mrad or at least about 0.25, 0.5, 0.75. 1.0, 1.5, 2.0, 2.5, 3.0, 3.5, 4.0, or at least about 5.0 Mrad. In some embodiments, the irradiation is performed until the material receives a dose of between 0.1 Mrad and 2.0 Mrad, e.g., between 0.5 Mrad and 4.0 Mrad or between 1.0 Mrad and 3.0 Mrad. It also can be desirable to irradiate from multiple directions, simultaneously or sequentially, in order to achieve a desired degree of penetration of radiation into the material. For example, depending on the density and moisture content of the material, such as wood, and the type of radiation source used (e.g., gamma or electron beam), the maximum penetration of radiation into the material may be only about 0.75 inch. In such cases, a thicker section (up to 1.5 inch) can be irradiated by first irradiating the material from one side, and then turning the material over and irradiating from the other side. Irradiation from multiple directions can be particularly useful with electron beam radiation, which irradiates faster than gamma radiation but typically does not achieve as great a penetration depth.

### Radiation Opaque Materials

The invention can include processing the material in a vault and/or bunker that is constructed using radiation opaque materials. In some implementations, the radiation opaque materials are selected to be capable of shielding the components from X-rays with high energy (short wavelength), which can penetrate many materials. One important factor in designing a radiation shielding enclosure is the attenuation length of the materials used, which will determine the required thickness for a particular material, blend of materials, or layered structure. The attenuation length is the penetration distance at which the radiation is reduced to approximately 1/e (e=Euler's number) times that of the incident radiation. Although virtually all materials are radiation opaque if thick enough, materials containing a high compositional percentage (e.g., density) of elements that have a high Z value (atomic number) have a shorter radiation attenuation length and thus if such materials are used a thinner, lighter shielding can be provided. Examples of high Z value materials that are used in radiation shielding are tantalum and lead. Another important parameter in radiation shielding is the halving distance, which is the thickness of a particular material that will reduce gamma ray intensity by 50%. As an example for X-ray radiation with an energy of 0.1 MeV the halving thickness is about 15.1 mm for concrete and about 2.7 mm for lead, while with an X-ray energy of 1 MeV the halving thickness for concrete is about 44.45 mm and for lead is about 7.9 mm Radiation opaque materials can be materials that are thick or thin so long as they can reduce the radiation that passes through to the other side. Thus, if it is desired that a particular enclosure have a low wall thickness, e.g., for light weight or due to size constraints, the material chosen should have a sufficient Z value and/or attenuation length so that its halving length is less than or equal to the desired wall thickness of the enclosure.

In some cases, the radiation opaque material may be a layered material. For example, having a layer of a higher Z value material, to provide good shielding, and a layer of a lower Z value material to provide other properties (e.g., structural integrity, impact resistance, etc.). In some cases, the layered material may be a “graded-Z” laminate, e.g., including a laminate in which the layers provide a gradient from high-Z through successively lower-Z elements. In some cases the radiation opaque materials can be interlocking blocks, for example, lead and/or concrete blocks can be supplied by NELCO Worldwide (Burlington, Mass.), and reconfigurable vaults can be utilized.

A radiation opaque material can reduce the radiation passing through a structure (e.g., a wall, door, ceiling, enclosure, a series of these or combinations of these) formed of the material by about at least about 10%, (e.g., at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%) as compared to the incident radiation. Therefore, an enclosure made of a radiation opaque material can reduce the exposure of equipment/system/components by the same amount. Radiation opaque materials can include stainless steel, metals with Z values above 25 (e.g., lead, iron), concrete, dirt, sand and combinations thereof. Radiation opaque materials can include a barrier in the direction of the incident radiation of at least about 1 mm (e.g., 5 mm, 10 mm, 5 cm, 10 cm, 100 cm, 1 m and even at least about 10 m).

### Radiation Sources

The type of radiation determines the kinds of radiation sources used as well as the radiation devices and associated equipment. The methods, systems and equipment described herein, for example, for treating materials with radiation, can be utilized sources as described herein as well as any other useful source.

Sources of gamma rays include radioactive nuclei, such as isotopes of cobalt, calcium, technetium, chromium, gallium, indium, iodine, iron, krypton, samarium, selenium, sodium, thallium, and xenon.

Sources of X-rays include electron beam collision with metal targets, such as tungsten or molybdenum or alloys, or compact light sources, such as those produced commercially by Lyncean.

Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium.

Sources for ultraviolet radiation include deuterium or cadmium lamps.

Sources for infrared radiation include sapphire, zinc, or selenide window ceramic lamps.

Sources for microwaves include klystrons, Slevin type RF sources, or atom beam sources that employ hydrogen, oxygen, or nitrogen gases.

Accelerators used to accelerate the particles (e.g., electrons or ions) can be DC (e.g., electrostatic DC or electrodynamic DC), RF linear, magnetic induction linear or continuous wave. For example, various irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, Cockroft Walton accelerators (e.g., PELLETRON® accelerators), LINACS, Dynamitrons (e.g., DYNAMITRON® accelerators), cyclotrons, synchrotrons, betatrons, transformer-type accelerators, microtrons, plasma generators, cascade accelerators, and folded tandem accelerators. For example, cyclotron type accelerators are available from IBA, Belgium, such as the RHODOTRON™ system, while DC type accelerators are available from RDI, now IBA Industrial, such as the DYNAMITRON®. Other suitable accelerator systems include, for example: DC insulated core transformer (ICT) type systems, available from Nissin High Voltage, Japan; S-band LINACs, available from L3-PSD (USA), Linac Systems (France), Mevex (Canada), and Mitsubishi Heavy Industries (Japan); L-band LINACs, available from Iotron Industries (Canada); and ILU-based accelerators, available from Budker Laboratories (Russia). Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 Mar. 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria. Some particle accelerators and their uses are disclosed, for example, in U.S. Pat. No. 7,931,784 to Medoff, the complete disclosure of which is incorporated herein by reference.

Electrons may be produced by radioactive nuclei that undergo beta decay, such as isotopes of iodine, cesium, technetium, and iridium. Alternatively, an electron gun can be used as an electron source via thermionic emission and accelerated through an accelerating potential. An electron gun generates electrons, which are then accelerated through a large potential (e.g., greater than about 500 thousand, greater than about 1 million, greater than about 2 million, greater than about 5 million, greater than about 6 million, greater than about 7 million, greater than about 8 million, greater than about 9 million, or even greater than 10 million volts) and then scanned magnetically in the x-y plane, where the electrons are initially accelerated in the z direction down the accelerator tube and extracted through a foil window. Scanning the electron beams is useful for increasing the irradiation surface when irradiating materials, e.g., a biomass, that is conveyed through the scanned beam. Scanning the electron beam also distributes the thermal load homogenously on the window and helps reduce the foil window rupture due to local heating by the electron beam. Window foil rupture is a cause of significant down-time due to subsequent necessary repairs and re-starting the electron gun.

Various other irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, and folded tandem accelerators. Such devices are disclosed, for example, in U.S. Pat. No. 7,931,784 to Medoff, the complete disclosure of which is incorporated herein by reference.

A beam of electrons can be used as the radiation source. A beam of electrons has the advantages of high dose rates (e.g., 1, 5, or even 10 Mrad per second), high throughput, less containment, and less confinement equipment. Electron beams can also have high electrical efficiency (e.g., 80%), allowing for lower energy usage relative to other radiation methods, which can translate into a lower cost of operation and lower greenhouse gas emissions corresponding to the smaller amount of energy used. Electron beams can be generated, e.g., by electrostatic generators, cascade generators, transformer generators, low energy accelerators with a scanning system, low energy accelerators with a linear cathode, linear accelerators, and pulsed accelerators.

Electrons can also be more efficient at causing changes in the molecular structure of carbohydrate-containing materials, for example, by the mechanism of chain scission. In addition, electrons having energies of 0.5-10 MeV can penetrate low density materials, such as the biomass materials described herein, e.g., materials having a bulk density of less than 0.5 g/cm³, and a depth of 0.3-10 cm. Electrons as an ionizing radiation source can be useful, e.g., for relatively thin piles, layers or beds of materials, e.g., less than about 0.5 inch, e.g., less than about 0.4 inch, 0.3 inch, 0.25 inch, or less than about 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV. Methods of irradiating materials are discussed in U.S. Pat. App. Pub. 2012/0100577 A1, filed Oct. 18, 2011, the entire disclosure of which is herein incorporated by reference.

Electron beam irradiation devices may be procured commercially or built. For example, elements or components such inductors, capacitors, casings, power sources, cables, wiring, voltage control systems, current control elements, insulating material, microcontrollers and cooling equipment can be purchased and assembled into a device. Optionally, a commercial device can be modified and/or adapted. For example, devices and components can be purchased from any of the commercial sources described herein including Ion Beam Applications (Louvain-la-Neuve, Belgium), Wasik Associates Inc. (Dracut, Mass.), NHV Corporation (Japan), the Titan Corporation (San Diego, Calif.), Vivirad High Voltage Corp (Billerica, Mass.) and/or Budker Laboratories (Russia). Typical electron energies can be 0.5 MeV, 1 MeV, 2 MeV, 4.5 MeV, 7.5 MeV, or 10 MeV. Typical electron beam irradiation device power can be 1 kW, 5 kW, 10 kW, 20 kW, 50 kW, 60 kW, 70 kW, 80 kW, 90 kW, 100 kW, 125 kW, 150 kW, 175 kW, 200 kW, 250 kW, 300 kW, 350 kW, 400 kW, 450 kW, 500 kW, 600 kW, 700 kW, 800 kW, 900 kW or even 1000 kW. Accelerators that can be used include NHV irradiators medium energy series EPS-500 (e.g., 500 kV accelerator voltage and 65, 100 or 150 mA beam current), EPS-800 (e.g., 800 kV accelerator voltage and 65 or 100 mA beam current), or EPS-1000 (e.g., 1000 kV accelerator voltage and 65 or 100 mA beam current). Also, accelerators from NHV's high energy series can be used such as EPS-1500 (e.g., 1500 kV accelerator voltage and 65 mA beam current), EPS-2000 (e.g., 2000 kV accelerator voltage and 50 mA beam current), EPS-3000 (e.g., 3000 kV accelerator voltage and 50 mA beam current) and EPS-5000 (e.g., 5000 and 30 mA beam current).

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Typically, generators are housed in a vault, e.g., of lead or concrete, especially for production from X-rays that are generated in the process. Tradeoffs in considering electron energies include energy costs.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available. The scanning beam is preferred in most embodiments described herein because of the larger scan width and reduced possibility of local heating and failure of the windows.

### Electron Guns—Windows

The extraction system for an electron accelerator can include two window foils. The cooling gas in the two foil window extraction system can be a purge gas or a mixture. For example, air, or a pure gas. In one embodiment the gas is an inert gas such as nitrogen, argon, helium and or carbon dioxide. It is preferred to use a gas rather than a liquid since energy losses to the electron beam are minimized. Mixtures of pure gas can also be used, either pre-mixed or mixed in line prior to impinging on the windows or in the space between the windows. The cooling gas can be cooled, for example, by using a heat exchange system (e.g., a chiller) and/or by using boil off from a condensed gas (e.g., liquid nitrogen, liquid helium). Window foils are described in PCT/US2013/64332 filed Oct. 10, 2013 the full disclosure of which is incorporated by reference herein.

### Heating and Throughput During Radiation Treatment

Several processes can occur in biomass when electrons from an electron beam interact with matter in inelastic collisions. For example, ionization of the material, chain scission of polymers in the material, cross linking of polymers in the material, oxidation of the material, generation of X-rays (“Bremsstrahlung”) and vibrational excitation of molecules (e.g., phonon generation). Without being bound to a particular mechanism, the reduction in recalcitrance can be due to several of these inelastic collision effects. For example, ionization, chain scission of polymers, oxidation and phonon generation. Some of the effects (e.g., especially X-ray generation), necessitate shielding and engineering barriers, for example, enclosing the irradiation processes in a concrete (or other radiation opaque material) vault. Another effect of irradiation, vibrational excitation, is equivalent to heating up the sample. Heating the sample by irradiation can help in recalcitrance reduction, but excessive heating can destroy the material, as will be explained below.

The adiabatic temperature rise (ΔT) from adsorption of ionizing radiation is given by the equation: ΔT=D/Cp: where D is the average dose in kGy, Cp is the heat capacity in J/g ° C., and ΔT is the change in temperature in ° C. A typical dry biomass material will have a heat capacity close to 2. Wet biomass will have a higher heat capacity dependent on the amount of water since the heat capacity of water is very high (4.19 J/g ° C.). Metals have much lower heat capacities, For example, 304 stainless steel has a heat capacity of 0.5 J/g ° C. The temperature change due to the instant adsorption of radiation in a biomass and stainless steel for various doses of radiation is shown in Table 1. At the higher temperatures biomass will decompose causing extreme deviation from the estimated changes in temperature.

TABLE 1
Calculated Temperature increase for biomass and stainless steel.

| Dose (Mrad) | Estimated Biomass ΔT (° C.) | | Steel ΔT (° C.) |
|---|---|---|---|
| 10 | 50 | | 200 |
| 50 | 250 | (Decomposed) | 1000 |
| 100 | 500 | (Decomposed) | 2000 |
| 150 | 750 | (Decomposed) | 3000 |
| 200 | 1000 | (Decomposed) | 4000 |

High temperatures can destroy and or modify the biopolymers in biomass so that the polymers (e.g., cellulose) are unsuitable for further processing. A biomass subjected to high temperatures can become dark, sticky and give off odors indicating decomposition. The stickiness can even make the material hard to convey. The odors can be unpleasant and be a safety issue. In fact, keeping the biomass below about 200° C. has been found to be beneficial in the processes described herein (e.g., below about 190° C., below about 180° C., below about 170° C., below about 160° C., below about 150° C., below about 140° C., below about 130° C., below about 120° C., below about 110° C., between about 60° C. and 180° C., between about 60° C. and 160° C., between about 60° C. and 150° C., between about 60° C. and 140° C., between about 60° C. and 130° C., between about 60° C. and 120° C., between about 80° C. and 180° C., between about 100° C. and 180° C., between about 120° C. and 180° C., between about 140° C. and 180° C., between about 160° C. and 180° C., between about 100° C. and 140° C., between about 80° C. and 120° C.).

It has been found that irradiation above about 10 Mrad is desirable for the processes described herein (e.g., reduction of recalcitrance). A high throughput is also desirable so that the irradiation does not become a bottle neck in processing the biomass. The treatment is governed by a Dose rate equation: M=FP/D·time, where M is the mass of irradiated material (kg), F is the fraction of power that is adsorbed (unit less), P is the emitted power (kW=Voltage in MeV×Current in mA), time is the treatment time (sec) and D is the adsorbed dose (kGy). In an exemplary process where the fraction of adsorbed power is fixed, the Power emitted is constant and a set dosage is desired, the throughput (e.g., M, the biomass processed) can be increased by increasing the irradiation time. However, increasing the irradiation time without allowing the material to cool, can excessively heat the material as exemplified by the calculations shown above. Since biomass has a low thermal conductivity (less than about 0.1 Wm⁻¹K⁻¹), heat dissipation is slow, unlike. For example, metals (greater than about 10 Wm⁻¹K⁻¹) which can dissipate energy quickly as long as there is a heat sink to transfer the energy to.

### Electron Guns—Beam Stops

In some embodiments the systems and methods include a beam stop (e.g., a shutter). For example, the beam stop can be used to quickly stop or reduce the irradiation of material without powering down the electron beam device. Alternatively the beam stop can be used while powering up the electron beam, e.g., the beam stop can stop the electron beam until a beam current of a desired level is achieved. The beam stop can be placed between the primary foil window and a secondary foil window. For example, the beam stop can be mounted so that it is movable, that is, so that it can be moved into and out of the beam path. Even partial coverage of the beam can be used, for example, to control the dose of irradiation. The beam stop can be mounted to the floor, to a conveyor for the biomass, to a wall, to the radiation device (e.g., at the scan horn), or to any structural support. Preferably the beam stop is fixed in relation to the scan horn so that the beam can be effectively controlled by the beam stop. The beam stop can incorporate a hinge, a rail, wheels, slots, or other means allowing for its operation in moving into and out of the beam. The beam stop can be made of any material that will stop at least 5% of the electrons, e.g., at least 10%, 20%, 30%, 40%, 50%, 60%, 70%, at least 80%, 85%, 90%, 91%, 92%, 93%, 94%, 95%, 96%, 97%, 98%, 99% or even about 100% of the electrons.

The beam stop can be made of a metal including, but not limited to, stainless steel, lead, iron, molybdenum, silver, gold, titanium, aluminum, tin, or alloys of these, or laminates (layered materials) made with such metals (e.g., metal-coated ceramic, metal-coated polymer, metal-coated composite, multilayered metal materials).

The beam stop can be cooled, for example, with a cooling fluid such as an aqueous solution or a gas. The beam stop can be partially or completely hollow. For example, with cavities. Interior spaces of the beam stop can be used for cooling fluids and gases. The beam stop can be of any shape, including flat, curved, round, oval, square, rectangular, beveled and wedged shapes.

The beam stop can have perforations so as to allow some electrons through, thus controlling (e.g., reducing) the levels of radiation across the whole area of the window, or in specific regions of the window. The beam stop can be a mesh formed, for example, from fibers or wires. Multiple beam stops can be used, together or independently, to control the irradiation. The beam stop can be remotely controlled, e.g., by radio signal or hard wired to a motor for moving the beam into or out of position.

### Beam Dumps

The embodiments disclosed herein can also include a beam dump when utilizing a radiation treatment. A beam dump's purpose is to safely absorb a beam of charged particles. Like a beam stop, a beam dump can be used to block the beam of charged particles. However, a beam dump is much more robust than a beam stop, and is intended to block the full power of the electron beam for an extended period of time. They are often used to block the beam as the accelerator is powering up.

Beam dumps are also designed to accommodate the heat generated by such beams, and are usually made from materials such as copper, aluminum, carbon, beryllium, tungsten, or mercury. Beam dumps can be cooled, for example, using a cooling fluid that can be in thermal contact with the beam dump.

### Biomass Materials

Lignocellulosic materials include, but are not limited to, wood, particle board, forestry wastes (e.g., sawdust, aspen wood, wood chips), grasses, (e.g., switchgrass, miscanthus, cord grass, reed canary grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair), sugar processing residues (e.g., bagasse, beet pulp, agave bagasse), algae, seaweed, manure, sewage, and mixtures of any of these.

In some cases, the lignocellulosic material includes corncobs. Ground or hammermilled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or cellulosic or lignocellulosic materials containing significant amounts of corncobs.

Corncobs, before and after comminution, are also easier to convey and disperse, and have a lesser tendency to form explosive mixtures in air than other cellulosic or lignocellulosic materials such as hay and grasses.

Cellulosic materials include, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high α-cellulose content such as cotton, and mixtures of any of these. For example, paper products as described in U.S. application Ser. No. 13/396,365 (“Magazine Feedstocks” by Medoff et al., filed Feb. 14, 2012), the full disclosure of which is incorporated herein by reference.

Cellulosic materials can also include lignocellulosic materials which have been partially or fully de-lignified.

In some instances other biomass materials can be utilized. For example, starchy materials. Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, ocra, sago, sorghum, regular household potatoes, sweet potato, taro, yams, or one or more beans, such as favas, lentils or peas. Blends of any two or more starchy materials are also starchy materials. Mixtures of starchy, cellulosic and or lignocellulosic materials can also be used. For example, a biomass can be an entire plant, a part of a plant or different parts of a plant, e.g., a wheat plant, cotton plant, a corn plant, rice plant or a tree. The starchy materials can be treated by any of the methods described herein.

Microbial materials that can be used as feedstock can include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

In other embodiments, the biomass materials, such as cellulosic, starchy and lignocellulosic feedstock materials, can be obtained from transgenic microorganisms and plants that have been modified with respect to a wild type variety. Such modifications may be, for example, through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. application Ser. No. 13/396,369 filed Feb. 14, 2012 the full disclosure of which is incorporated herein by reference.

Any of the methods described herein can be practiced with mixtures of any biomass materials described herein.

### Other Materials

Other materials (e.g., natural or synthetic materials), for example, polymers, can be treated and/or made utilizing the methods, equipment and systems described herein. For example, polyethylene (e.g., linear low density ethylene and high density polyethylene), polystyrenes, sulfonated polystyrenes, poly (vinyl chloride), polyesters (e.g., nylons, DACRON™, KODEL™), polyalkylene esters, poly vinyl esters, polyamides (e.g., KEVLAR™), polyethylene terephthalate, cellulose acetate, acetal, poly acrylonitrile, polycarbonates (e.g., LEXAN™), acrylics [e.g., poly (methyl methacrylate), poly(methyl methacrylate), polyacrylonitrile], Poly urethanes, polypropylene, poly butadiene, polyisobutylene, polyacrylonitrile, polychloroprene (e.g. neoprene), poly(cis-1,4-isoprene) [e.g., natural rubber], poly(trans-1,4-isoprene) [e.g., gutta percha], phenol formaldehyde, melamine formaldehyde, epoxides, polyesters, poly amines, polycarboxylic acids, polylactic acids, polyvinyl alcohols, polyanhydrides, poly fluoro carbons (e.g., TEFLON™), silicons (e.g., silicone rubber), polysilanes, poly ethers (e.g., polyethylene oxide, polypropylene oxide), waxes, oils and mixtures of these. Also included are plastics, rubbers, elastomers, fibers, waxes, gels, oils, adhesives, thermoplastics, thermosets, biodegradable polymers, resins made with these polymers, other polymers, other materials and combinations thereof. The polymers can be made by any useful method including cationic polymerization, anionic polymerization, radical polymerization, metathesis polymerization, ring opening polymerization, graft polymerization, addition polymerization. In some cases the treatments disclosed herein can be used, for example, for radically initiated graft polymerization and cross linking. Composites of polymers, for example, with glass, metals, biomass (e.g., fibers, particles), ceramics can also be treated and/or made.

Other materials that can be treated by using the methods, systems and equipment disclosed herein are ceramic materials, minerals, metals, inorganic compounds. For example, silicon and germanium crystals, silicon nitrides, metal oxides, semiconductors, insulators, cements and or conductors.

In addition, manufactured multipart or shaped materials (e.g., molded, extruded, welded, riveted, layered or combined in any way) can be treated. For example, cables, pipes, boards, enclosures, integrated semiconductor chips, circuit boards, wires, tires, windows, laminated materials, gears, belts, machines, combinations of these. For example, treating a material by the methods described herein can modify the surfaces, for example, making them susceptible to further functionalization, combinations (e.g., welding) and/or treatment can cross link the materials.

### Biomass Material Preparation—Mechanical Treatments

The biomass can be in a dry form. For example, with less than about 35% moisture content (e.g., less than about 20%, less than about 15%, less than about 10% less than about 5%, less than about 4%, less than about 3%, less than about 2% or even less than about 1%). The biomass can also be delivered in a wet state. For example, as a wet solid, a slurry or a suspension with at least about 10 wt % solids (e.g., at least about 20 wt. %, at least about 30 wt. %, at least about 40 wt. %, at least about 50 wt. %, at least about 60 wt. %, at least about 70 wt. %).

The processes disclosed herein can utilize low bulk density materials. For example, cellulosicor lignocellulosic feedstocks that have been physically pretreated to have a bulk density of less than about 0.75 g/cm³, e.g., less than about 0.7, 0.65, 0.60, 0.50, 0.35, 0.25, 0.20, 0.15, 0.10, 0.05 or less, e.g., less than about 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters. If desired, low bulk density materials can be densified, for example, by methods described in U.S. Pat. No. 7,971,809 to Medoff, the full disclosure of which is hereby incorporated by reference.

In some cases, the pre-treatment processing includes screening of the biomass material. Screening can be through a mesh or perforated plate with a desired opening size, for example, less than about 6.35 mm (¼ inch, 0.25 inch), (e.g., less than about 3.18 mm (⅛ inch, 0.125 inch), less than about 1.59 mm ( 1/16 inch, 0.0625 inch), is less than about 0.79 mm ( 1/32 inch, 0.03125 inch), e.g., less than about 0.51 mm ( 1/50 inch, 0.02000 inch), less than about 0.40 mm ( 1/64 inch, 0.015625 inch), less than about 0.23 mm (0.009 inch), less than about 0.20 mm ( 1/128 inch, 0.0078125 inch), less than about 0.18 mm (0.007 inch), less than about 0.13 mm (0.005 inch), or even less than about 0.10 mm ( 1/256 inch, 0.00390625 inch)). In one configuration the desired biomass falls through the perforations or screen and thus biomass larger than the perforations or screen are not irradiated. These larger materials can be re-processed, for example, by comminuting, or they can simply be removed from processing. In another configuration material that is larger than the perforations is irradiated and the smaller material is removed by the screening process or recycled. In this kind of a configuration, the conveyor itself (for example, a part of the conveyor) can be perforated or made with a mesh. For example, in one particular embodiment the biomass material may be wet and the perforations or mesh allow water to drain away from the biomass before irradiation.

Screening of material can also be by a manual method, for example, by an operator or mechanoid (e.g., a robot equipped with a color, reflectivity or other sensor) that removes unwanted material. Screening can also be by magnetic screening wherein a magnet is disposed near the conveyed material and the magnetic material is removed magnetically.

Optional pre-treatment processing can include heating the material. For example, a portion of a conveyor conveying the biomass or other material can be sent through a heated zone. The heated zone can be created, for example, by IR radiation, microwaves, combustion (e.g., gas, coal, oil, biomass), resistive heating and/or inductive coils. The heat can be applied from at least one side or more than one side, can be continuous or periodic and can be for only a portion of the material or all the material. For example, a portion of the conveying trough can be heated by use of a heating jacket. Heating can be, for example, for the purpose of drying the material. In the case of drying the material, this can also be facilitated, with or without heating, by the movement of a gas (e.g., air, oxygen, nitrogen, He, CO₂, Argon) over and/or through the biomass as it is being conveyed.

Optionally, pre-treatment processing can include cooling the material. Cooling material is described in U.S. Pat. No. 7,900,857 to Medoff, the disclosure of which in incorporated herein by reference. For example, cooling can be by supplying a cooling fluid. For example, water (e.g., with glycerol), or nitrogen (e.g., liquid nitrogen) to the bottom of the conveying trough. Alternatively, a cooling gas, for example, chilled nitrogen can be blown over the biomass materials or under the conveying system.

Another optional pre-treatment processing method can include adding a material to the biomass or other feedstocks. The additional material can be added by, for example, by showering, sprinkling and or pouring the material onto the biomass as it is conveyed. Materials that can be added include, for example, metals, ceramics and/or ions as described in U.S. Pat. App. Pub. 2010/0105119 A1 (filed Oct. 26, 2009) and U.S. Pat. App. Pub. 2010/0159569 A1 (filed Dec. 16, 2009), the entire disclosures of which are incorporated herein by reference. Optional materials that can be added include acids and bases. Other materials that can be added are oxidants (e.g., peroxides, chlorates), polymers, polymerizable monomers (e.g., containing unsaturated bonds), water, catalysts, enzymes and/or organisms. Materials can be added, for example, in pure form, as a solution in a solvent (e.g., water or an organic solvent) and/or as a solution. In some cases the solvent is volatile and can be made to evaporate e.g., by heating and/or blowing gas as previously described. The added material may form a uniform coating on the biomass or be a homogeneous mixture of different components (e.g., biomass and additional material). The added material can modulate the subsequent irradiation step by increasing the efficiency of the irradiation, damping the irradiation or changing the effect of the irradiation (e.g., from electron beams to X-rays or heat). The method may have no impact on the irradiation but may be useful for further downstream processing. The added material may help in conveying the material, for example, by lowering dust levels.

Biomass can be delivered to a conveyor (e.g., vibratory conveyors used in the vaults herein described) by a belt conveyor, a pneumatic conveyor, a screw conveyor, a hopper, a pipe, manually or by a combination of these. The biomass can, for example, be dropped, poured and/or placed onto the conveyor by any of these methods. In some embodiments, the material is delivered to the conveyor using an enclosed material distribution system to help maintain a low oxygen atmosphere and/or control dust and fines. Lofted or air suspended biomass fines and dust are undesirable because these can form an explosion hazard or damage the window foils of an electron gun (if such a device is used for treating the material).

The material can be leveled to form a uniform thickness between about 0.0312 and 5 inches (e.g., between about 0.0625 and 2.000 inches, between about 0.125 and 1 inches, between about 0.125 and 0.5 inches, between about 0.3 and 0.9 inches, between about 0.2 and 0.5 inches between about 0.25 and 1.0 inches, between about 0.25 and 0.5 inches, 0.100+/−0.025 inches, 0.150+/−0.025 inches, 0.200+/−0.025 inches, 0.250+/−0.025 inches, 0.300+/−0.025 inches, 0.350+/−0.025 inches, 0.400+/−0.025 inches, 0.450+/−0.025 inches, 0.500+/−0.025 inches, 0.550+/−0.025 inches, 0.600+/−0.025 inches, 0.700+/−0.025 inches, 0.750+/−0.025 inches, 0.800+/−0.025 inches, 0.850+/−0.025 inches, 0.900+/−0.025 inches, 0.900+/−0.025 inches.

Generally, it is preferred to convey the material as quickly as possible through the electron beam to maximize throughput. For example, the material can be conveyed at rates of at least 1 ft/min, e.g., at least 2 ft/min, at least 3 ft/min, at least 4 ft/min, at least 5 ft/min, at least 10 ft/min, at least 15 ft/min, 20, 25, 30, 35, 40, 45, 50 ft/min. The rate of conveying is related to the beam current, for example, for a ¼ inch thick biomass and 100 mA, the conveyor can move at about 20 ft/min to provide a useful irradiation dosage, at 50 mA the conveyor can move at about 10 ft/min to provide approximately the same irradiation dosage.

After the biomass material has been conveyed through the radiation zone, optional post-treatment processing can be done. The optional post-treatment processing can, for example, be a process described with respect to the pre-irradiation processing. For example, the biomass can be screened, heated, cooled, and/or combined with additives. Uniquely to post-irradiation, quenching of the radicals can occur, for example, quenching of radicals by the addition of fluids or gases (e.g., oxygen, nitrous oxide, ammonia, liquids), using pressure, heat, and/or the addition of radical scavengers. For example, the biomass can be conveyed out of the enclosed conveyor and exposed to a gas (e.g., oxygen) where it is quenched, forming carboxylated groups. In one embodiment the biomass is exposed during irradiation to the reactive gas or fluid. Quenching of biomass that has been irradiated is described in U.S. Pat. No. 8,083,906 to Medoff, the entire disclosure of which is incorporated herein by reference.

If desired, one or more mechanical treatments can be used in addition to irradiation to further reduce the recalcitrance of the carbohydrate-containing material. These processes can be applied before, during and or after irradiation.

In some cases, the mechanical treatment may include an initial preparation of the feedstock as received, e.g., size reduction of materials, such as by comminution, e.g., cutting, grinding, shearing, pulverizing or chopping. For example, in some cases, loose feedstock (e.g., recycled paper, starchy materials, or switchgrass) is prepared by shearing or shredding. Mechanical treatment may reduce the bulk density of the carbohydrate-containing material, increase the surface area of the carbohydrate-containing material and/or decrease one or more dimensions of the carbohydrate-containing material.

Alternatively, or in addition, the feedstock material can be treated with another treatment. For example, chemical treatments, such as an with an acid (HCl, H₂SO₄, H₃PO₄), a base (e.g., KOH and NaOH), a chemical oxidant (e.g., peroxides, chlorates, ozone), irradiation, steam explosion, pyrolysis, sonication, oxidation, chemical treatment. The treatments can be in any order and in any sequence and combinations. For example, the feedstock material can first be physically treated by one or more treatment methods, e.g., chemical treatment including and in combination with acid hydrolysis (e.g., utilizing HCl, H₂SO₄, H₃PO₄), radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the structure of the material by mechanical treatment. As another example, a feedstock material can be conveyed through ionizing radiation using a conveyor as described herein and then mechanically treated. Chemical treatment can remove some or all of the lignin. (For example, chemical pulping) and can partially or completely hydrolyze the material. The methods also can be used with pre-hydrolyzed material. The methods also can be used with material that has not been pre hydrolyzed. The methods can be used with mixtures of hydrolyzed and non-hydrolyzed materials. For example, with about 50% or more non-hydrolyzed material, with about 60% or more non-hydrolyzed material, with about 70% or more non-hydrolyzed material, with about 80% or more non-hydrolyzed material or even with 90% or more non-hydrolyzed material.

In addition to size reduction, which can be performed initially and/or later in processing, mechanical treatment can also be advantageous for “opening up,” “stressing,” breaking or shattering the carbohydrate-containing materials, making the cellulose of the materials more susceptible to chain scission and/or disruption of crystalline structure during the physical treatment.

Methods of mechanically treating the carbohydrate-containing material include, for example, wet or dry milling, and/or wet or dry grinding. Milling may be performed using, for example, a hammer mill, ball mill, colloid mill, conical or cone mill, disk mill, edge mill, Wiley mill, grist mill, rotor/stator or other mill Grinding may be performed using, for example, a cutting/impact type grinder. Some exemplary grinders include stone grinders, pin grinders, coffee grinders, and burr grinders. Grinding or milling may be provided, for example, by a reciprocating pin or other element, as is the case in a pin mill Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps. Some milling method, such as utilizing a rotor/stator are described in International Publication No. WO 2010/009240, published on Jan. 21, 2010; International Publication No. WO 2011/090543, published on Jul. 28, 2011; and International Publication No. WO 2012/170707, published on Dec. 13, 2012, the full disclosure of each of these applications is incorporated by reference herein.

Mechanical feed preparation systems can be configured to produce streams with specific characteristics such as, for example, specific maximum sizes, specific length-to-width, or specific surface areas ratios. Physical preparation can increase the rate of reactions, improve the movement of material on a conveyor, improve the irradiation profile of the material, improve the radiation uniformity of the material, or reduce the processing time required by opening up the materials and making them more accessible to processes and/or reagents, such as reagents in a solution.

The bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state (e.g., after transport). The material can be densified, for example, from less than about 0.2 g/cc to more than about 0.9 g/cc (e.g., less than about 0.3 to more than about 0.5 g/cc, less than about 0.3 to more than about 0.9 g/cc, less than about 0.5 to more than about 0.9 g/cc, less than about 0.3 to more than about 0.8 g/cc, less than about 0.2 to more than about 0.5 g/cc). For example, the material can be densified by the methods and equipment disclosed in U.S. Pat. No. 7,932,065 to Medoff and International Publication No. WO 2008/073186 (which was filed Oct. 26, 2007, was published in English, and which designated the United States), the full disclosures of which are incorporated herein by reference. Densified materials can be processed by any of the methods described herein, or any material processed by any of the methods described herein can be subsequently densified.

In some embodiments, the material to be processed is in the form of a fibrous material that includes fibers provided by shearing a fiber source. For example, the shearing can be performed with a rotary knife cutter.

For example, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process.

For example, a rotary knife cutter can be used to concurrently shear the fiber source and screen the first fibrous material. A rotary knife cutter includes a hopper that can be loaded with a shredded fiber source prepared by shredding a fiber source.

In some implementations, the feedstock is physically treated prior to saccharification and/or fermentation. Physical treatment processes can include one or more of any of those described herein, such as mechanical treatment, chemical treatment, irradiation, sonication, oxidation, pyrolysis or steam explosion. Treatment methods can be used in combinations of two, three, four, or even all of these technologies (in any order). When more than one treatment method is used, the methods can be applied at the same time or at different times. Other processes that change a molecular structure of a biomass feedstock may also be used, alone or in combination with the processes disclosed herein.

Mechanical treatments that may be used, and the characteristics of the mechanically treated carbohydrate-containing materials, are described in further detail in U.S. Pat. App. Pub. 2012/0100577 A1, filed Oct. 18, 2011, the full disclosure of which is hereby incorporated herein by reference.

### Sonication, Pyrolysis, Oxidation, Steam Explosion

If desired, one or more sonication, pyrolysis, oxidative, or steam explosion processes can be used instead of or in addition to irradiation to reduce or further reduce the recalcitrance of the carbohydrate-containing material. For example, these processes can be applied before, during and or after irradiation. These processes are described in detail in U.S. Pat. No. 7,932,065 to Medoff, the full disclosure of which is incorporated herein by reference.

### Heat Treatment of Biomass

Alternately, or in addition to the biomass may be heat treated for up to twelve hours at temperatures ranging from about 90° C. to about 160° C. Optionally, this heat treatment step is performed after biomass has been irradiated with an electron beam. The amount of time for the heat treatment is up to 9 hours, alternately up to 6 hours, optionally up to 4 hours and further up to about 2 hours. The treatment time can be up to as little as 30 minutes when the mass may be effectively heated.

The heat treatment can be performed 90° C. to about 160° C. or, optionally, at 100 to 150 or, alternatively, at 120 to 140° C. The biomass is suspended in water such that the biomass content is 10 to 75 wt. % in water. In the case of the biomass being the irradiated biomass water is added and the heat treatment performed.

The heat treatment is performed in an aqueous suspension or mixture of the biomass. The amount of biomass is 10 to 90 wt. % of the total mixture, alternatively 20 to 70 wt. % or optionally 25 to 50 wt. %. The irradiated biomass may have minimal water content so water must be added prior to the heat treatment.

Since at temperatures above 100 Deg C there will be pressure due at least in part to the vaporization of water, a pressure vessel can be utilized to accommodate and/or maintain the pressure. The process for the heat treatment may be batch, continuous, semi-continuous or other reactor configurations. The continuous reactor configuration may be a tubular reactor and may include device(s) within the tube which will facilitate heat transfer and mixing/suspension of the biomass. These tubular devices may include a one or more static mixers. The heat may also be put into the system by direct injection of steam.

### Intermediates and Products

Using the processes described herein, the biomass material can be converted to one or more products, such as energy, fuels, foods and materials. For example, intermediates and products such as organic acids, salts of organic acids, anhydrides, esters of organic acids and fuels, e.g., fuels for internal combustion engines or feedstocks for fuel cells. Systems and processes are described herein that can use as feedstock cellulosic and/or lignocellulosic materials that are readily available, but often can be difficult to process, e.g., municipal waste streams and waste paper streams, such as streams that include newspaper, Kraft paper, corrugated paper or mixtures of these.

Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols (e.g., containing greater than 10%, 20%, 30% or even greater than 40% water), biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives (e.g., fuel additives). Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha and beta unsaturated acids (e.g., acrylic acid) and olefins (e.g., ethylene). Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), and methyl or ethyl esters of any of these alcohols. Other products include methyl acrylate, methylmethacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, gamma-hydroxybutyric acid, and mixtures thereof, salts of any of these acids, mixtures of any of the acids and their respective salts.

Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be sanitized or sterilized prior to selling the products, e.g., after purification or isolation or even after packaging, to neutralize one or more potentially undesirable contaminants that could be present in the product(s). Such sanitation can be done with electron bombardment, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

Other intermediates and products, including food and pharmaceutical products, are described in U.S. Pat. App. Pub. 2010/0124583 A1, published May 20, 2010, to Medoff, the full disclosure of which is hereby incorporated by reference herein.

### Lignin Derived Products

The spent biomass (e.g., spent lignocellulosic material) from lignocellulosic processing by the methods described are expected to have a high lignin content, and in addition to being useful for producing energy through combustion in a Co-Generation plant, may have uses as other valuable products. For example, the lignin can be used as captured as a plastic, or it can be synthetically upgraded to other plastics. In some instances, it can also be converted to lignosulfonates, which can be utilized as binders, dispersants, emulsifiers or sequestrants.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

When used as a dispersant, the lignin or lignosulfonates can be used. For example, in concrete mixes, clay and ceramics, dyes and pigments, leather tanning and in gypsum board.

When used as an emulsifier, the lignin or lignosulfonates can be used, e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

For energy production, lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

### Saccharification

In order to convert the feedstock to a form that can be readily processed, the glucan- or xylan-containing cellulose in the feedstock can be hydrolyzed to low molecular weight carbohydrates, such as sugars, by a saccharifying agent, e.g., an enzyme or acid, a process referred to as saccharification. The low molecular weight carbohydrates can then be used, for example, in an existing manufacturing plant, such as a single cell protein plant, an enzyme manufacturing plant, or a fuel plant, e. g., an ethanol manufacturing facility.

The feedstock can be hydrolyzed using an enzyme, e.g., by combining the materials and the enzyme in a solvent, e.g., in an aqueous solution.

Alternatively, the enzymes can be supplied by organisms that break down biomass, such as the cellulose and/or the lignin portions of the biomass, contain or manufacture various cellulolytic enzymes (cellulases), ligninases or various small molecule biomass-degrading metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (beta-glucosidases).

During saccharification, a cellulosic substrate can be initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose. The efficiency (e.g., time to hydrolyze and/or completeness of hydrolysis) of this process depends on the recalcitrance of the cellulosic material.

Therefore, the treated biomass materials can be saccharified, generally by combining the material and a cellulase enzyme in a fluid medium, e.g., an aqueous solution. In some cases, the material is boiled, steeped, or cooked in hot water prior to saccharification, as described in U.S. Pat. App. Pub. 2012/0100577 A1 by Medoff and Masterman, published on Apr. 26, 2012, the entire contents of which are incorporated herein.

The saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. The time required for complete saccharification will depend on the process conditions and the carbohydrate-containing material and enzyme used. If saccharification is performed in a manufacturing plant under controlled conditions, the cellulose may be substantially entirely converted to sugar, e.g., glucose in about 12-96 hours. If saccharification is performed partially or completely in transit, saccharification may take longer.

It is generally preferred that the tank contents be mixed during saccharification, e.g., using jet mixing as described in International App. No. PCT/US2010/035331, filed May 18, 2010, which was published in English as WO 2010/135380 and designated the United States, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, or amphoteric surfactants.

It is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. This reduces the volume to be shipped, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. Preferably the antimicrobial additive(s) are food-grade.

A relatively high concentration solution can be obtained by limiting the amount of water added to the carbohydrate-containing material with the enzyme. The concentration can be controlled, e.g., by controlling how much saccharification takes place. For example, concentration can be increased by adding more carbohydrate-containing material to the solution. In order to keep the sugar that is being produced in solution, a surfactant can be added, e.g., one of those discussed above. Solubility can also be increased by increasing the temperature of the solution. For example, the solution can be maintained at a temperature of 40-50° C., 60-80° C., or even higher.

### Saccharifying Agents

Suitable cellulolytic enzymes include cellulases from species in the genera Bacillus, Coprinus, Myceliophthora, Cephalosporium, Scytalidium, Penicillium, Aspergillus, Pseudomonas, Humicola, Fusarium, Thielavia, Acremonium, Chrysosporium and Trichoderma, especially those produced by a strain selected from the species Aspergillus (see, e.g., EP Pub. No. 0 458 162), Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp. (including, but not limited to, A. persicinum, A. acremonium, A. brachypenium, A. dichromosporum, A. obclavatum, A. pinkertoniae, A. roseogriseum, A. incoloratum, and A. furatum). Preferred strains include Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additional strains that can be used include, but are not limited to, Trichoderma (particularly T. viride, T. reesei, and T. koningii), alkalophilic Bacillus (see, for example, U.S. Pat. No. 3,844,890 and EP Pub. No. 0 458 162), and Streptomyces (see, e.g., EP Pub. No. 0 458 162).

In addition to or in combination to enzymes, acids, bases and other chemicals (e.g., oxidants) can be utilized to saccharify lignocellulosic and cellulosic materials. These can be used in any combination or sequence (e.g., before, after and/or during addition of an enzyme). For example, strong mineral acids can be utilized (e.g. HCl, H₂SO₄, H₃PO₄) and strong bases (e.g., NaOH, KOH).

### Sugars

In the processes described herein, for example, after saccharification, sugars (e.g., glucose and xylose) can be isolated. For example, sugars can be isolated by precipitation, crystallization, chromatography (e.g., simulated moving bed chromatography, high pressure chromatography), centrifugation, extraction, any other isolation method known in the art, and combinations thereof.

### Hydrogenation and Other Chemical Transformations

The processes described herein can include hydrogenation. For example, glucose and xylose can be hydrogenated to sorbitol and xylitol respectively. Hydrogenation can be accomplished by use of a catalyst (e.g., Pt/gamma-Al₂O₃, Ru/C, Raney Nickel, or other catalysts know in the art) in combination with H₂ under high pressure (e.g., 10 to 12000 psi). Other types of chemical transformation of the products from the processes described herein can be used, for example, production of organic sugar derived products such (e.g., furfural and furfural-derived products). Chemical transformations of sugar derived products are described in U.S. Ser. No. 13/934,704 filed Jul. 3, 2013, the entire disclosure of which is incorporated herein by reference in its entirety.

### Fermentation

Yeast and Zymomonas bacteria, for example, can be used for fermentation or conversion of sugar(s) to alcohol(s). Other microorganisms are discussed below. The optimum pH for fermentations is about pH 4 to 7. For example, the optimum pH for yeast is from about pH 4 to 5, while the optimum pH for Zymomonas is from about pH 5 to 6. Typical fermentation times are about 24 to 168 hours (e.g., 24 to 96 hrs) with temperatures in the range of 20° C. to 40° C. (e.g., 26° C. to 40° C.), however thermophilic microorganisms prefer higher temperatures.

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic conditions can be achieved or maintained by carbon dioxide production during the fermentation and no additional inert gas is needed.

In some embodiments, all or a portion of the fermentation process can be interrupted before the low molecular weight sugar is completely converted to a product (e.g., ethanol). The intermediate fermentation products include sugar and carbohydrates in high concentrations. The sugars and carbohydrates can be isolated via any means known in the art. These intermediate fermentation products can be used in preparation of food for human or animal consumption. Additionally or alternatively, the intermediate fermentation products can be ground to a fine particle size in a stainless-steel laboratory mill to produce a flour-like substance. Jet mixing may be used during fermentation, and in some cases saccharification and fermentation are performed in the same tank.

Nutrients for the microorganisms may be added during saccharification and/or fermentation, for example, the food-based nutrient packages described in U.S. Pat. App. Pub. 2012/0052536, filed Jul. 15, 2011, the complete disclosure of which is incorporated herein by reference.

“Fermentation” includes the methods and products that are disclosed in application Nos. PCT/US2012/71093 published Jun. 27, 2013, PCT/US2012/71907 published Jun. 27, 2012, and PCT/US2012/71083 published Jun. 27, 2012 the contents of which are incorporated by reference herein in their entirety.

Mobile fermenters can be utilized, as described in International App. No. PCT/US2007/074028 (which was filed Jul. 20, 2007, was published in English as WO 2008/011598 and designated the United States) and has a U.S. issued U.S. Pat. No. 8,318,453, the contents of which are incorporated herein in its entirety. Similarly, the saccharification equipment can be mobile. Further, saccharification and/or fermentation may be performed in part or entirely during transit.

### Fermentation Agents

The microorganism(s) used in fermentation can be naturally-occurring microorganisms and/or engineered microorganisms. For example, the microorganism can be a bacterium (including, but not limited to, e.g., a cellulolytic bacterium), a fungus, (including, but not limited to, e.g., a yeast), a plant, a protist, e.g., a protozoa or a fungus-like protest (including, but not limited to, e.g., a slime mold), or an alga. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K. fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose bioconversion technology, in Handbook on Bioethanol: Production and Utilization, Wyman, C. E., ed., Taylor & Francis, Washington, D.C., 179-212)). Other suitable microorganisms include, for example, Zymomonas mobilis, Clostridium spp. (including, but not limited to, C. thermocellum (Philippidis, 1996, supra), C. saccharobutylacetonicum, C. tyrobutyricum C. saccharobutylicum, C. Puniceum, C. beijernckii, and C. acetobutylicum), Moniliella spp. (including but not limited to M. pollinis, M. tomentosa, M. madida, M. nigrescens, M. oedocephali, M. megachiliensis), Yarrowia lipolytica, Aureobasidium sp., Trichosporonoides sp., Trigonopsis variabilis, Trichosporon sp., Moniliellaacetoabutans sp., Typhula variabilis, Candida magnoliae, Ustilaginomycetes sp., Pseudozyma tsukubaensis, yeast species of genera Zygosaccharomyces, Debaryomyces, Hansenula and Pichia, and fungi of the dematioid genus Torula (e.g., T. corallina).

Many such microbial strains are publicly available, either commercially or through depositories such as the ATCC (American Type Culture Collection, Manassas, Va., USA), the NRRL (Agricultural Research Service Culture Collection, Peoria, Ill., USA), or the DSMZ (Deutsche Sammlung von Mikroorganismen and Zellkulturen GmbH, Braunschweig, Germany), to name a few.

Commercially available yeasts include, for example, RED STAR®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Hydrocarbon-Containing Materials

In other embodiments utilizing the methods and systems described herein, hydrocarbon-containing materials can be processed. Any process described herein can be used to treat any hydrocarbon-containing material herein described. “Hydrocarbon-containing materials,” as used herein, is meant to include oil sands, oil shale, tar sands, coal dust, coal slurry, bitumen, various types of coal, and other naturally-occurring and synthetic materials that include both hydrocarbon components and solid matter. The solid matter can include rock, sand, clay, stone, silt, drilling slurry, or other solid organic and/or inorganic matter. The term can also include waste products such as drilling waste and by-products, refining waste and by-products, or other waste products containing hydrocarbon components, such as asphalt shingling and covering, asphalt pavement, etc.

In yet other embodiments utilizing the methods and systems described herein, wood and wood containing produces can be processed. For example, lumber products can be processed, e.g. boards, sheets, laminates, beams, particle boards, composites, rough cut wood, soft wood and hard wood. In addition cut trees, bushes, wood chips, saw dust, roots, bark, stumps, decomposed wood and other wood containing biomass material can be processed.

### Conveying Systems

Various conveying systems can be used to convey the biomass material, for example, as discussed, to a vault, and under an electron beam in a vault. Exemplary conveyors are belt conveyors, pneumatic conveyors, screw conveyors, carts, trains, trains or carts on rails, elevators, front loaders, backhoes, cranes, various scrapers and shovels, trucks, and throwing devices can be used. For example, vibratory conveyors can be used in various processes described herein. Vibratory conveyors are described in PCT/US2013/64289 filed Oct. 10, 2013 the full disclosure of which is incorporated by reference herein.

Vibratory conveyors are particularly useful for spreading the material and producing a uniform layer on the conveyor trough surface. For example, the initial feedstock can form a pile of material that can be at least four feet high (e.g., at least about 3 feet, at least about 2 feet, at least about 1 foot, at least about 6 inches, at least about 5 inches, at least about, 4 inches, at least about 3 inches, at least about 2 inches, at least about 1 inch, at least about ½ inch) and spans less than the width of the conveyor (e.g., less than about 10%, less than about 20%, less than about 30%, less than about 40%, less than about 50%, less than about 60%, less than about 70%, less than about 80%, less than about 90%, less than about 95%, less than about 99%). The vibratory conveyor can spread the material to span the entire width of the conveyor trough and have a uniform thickness, preferably as discussed above. In some cases, an additional spreading method can be useful. For example, a spreader such as a broadcast spreader, a drop spreader (e.g., a CHRISTY SPREADER™) or combinations thereof can be used to drop (e.g., place, pour, spill and/or sprinkle) the feedstock over a wide area. Optionally, the spreader can deliver the biomass as a wide shower or curtain onto the vibratory conveyor. Additionally, a second conveyor, upstream from the first conveyor (e.g., the first conveyor is used in the irradiation of the feedstock), can drop biomass onto the first conveyor, where the second conveyor can have a width transverse to the direction of conveying smaller than the first conveyor. In particular, when the second conveyor is a vibratory conveyor, the feedstock is spread by the action of the second and first conveyor. In some optional embodiments, the second conveyor ends in a bias cross cut discharge (e.g., a bias cut with a ratio of 4:1) so that the material can be dropped as a wide curtain (e.g., wider than the width of the second conveyor) onto the first conveyor. The initial drop area of the biomass by the spreader (e.g., broadcast spreader, drop spreader, conveyor, or cross cut vibratory conveyor) can span the entire width of the first vibratory conveyor, or it can span part of this width. Once dropped onto the conveyor, the material is spread even more uniformly by the vibrations of the conveyor so that, preferably, the entire width of the conveyor is covered with a uniform layer of biomass. In some embodiments combinations of spreaders can be used. Some methods of spreading a feed stock are described in U.S. Pat. No. 7,153,533, filed Jul. 23, 2002 and published Dec. 26, 2006, the entire disclosure of which is incorporated herein by reference.

Generally, it is preferred to convey the material as quickly as possible through an electron beam to maximize throughput. For example, the material can be conveyed at rates of at least 1 ft/min, e.g., at least 2 ft/min, at least 3 ft/min, at least 4 ft/min, at least 5 ft/min, at least 10 ft/min, at least 15 ft/min, at least 20 ft/min, at least 25 ft/min, at least 30 ft/min, at least 40 ft/min, at least 50 ft/min, at least 60 ft/min, at least 70 ft/min, at least 80 ft/min, at least 90 ft/min. The rate of conveying is related to the beam current and targeted irradiation dose, for example, for a ¼ inch thick biomass spread over a 5.5 foot wide conveyor and 100 mA, the conveyor can move at about 20 ft/min to provide a useful irradiation dosage (e.g. about 10 Mrad for a single pass), at 50 mA the conveyor can move at about 10 ft/min to provide approximately the same irradiation dosage.

The rate at which material can be conveyed depends on the shape and mass of the material being conveyed. Flowing materials e.g., particulate materials, are particularly amenable to conveying with vibratory conveyors. Conveying speeds can, for example, be, at least 100 lb/hr (e.g., at least 500 lb/hr, at least 1000 lb/hr, at least 2000 lb/hr, at least 3000 lb/hr, at least 4000 lb/hr, at least 5000 lb/hr, at least 10,000 lb/hr, at least 15,000 lb/hr, or even at least 25,000 lb/hr). Some typical conveying speeds can be between about 1000 and 10,000 lb/hr, (e.g., between about 1000 lb/hr and 8000 lb/hr, between about 2000 and 7000 lb/hr, between about 2000 and 6000 lb/hr, between about 2000 and 5000 lb/hr, between about 2000 and 4500 lb/hr, between about 1500 and 5000 lb/hr, between about 3000 and 7000 lb/hr, between about 3000 and 6000 lb/hr, between about 4000 and 6000 lb/hr and between about 4000 and 5000 lb/hr). Typical conveying speeds depend on the density of the material. For example, for a biomass with a density of about 35 lb/ft3, and a conveying speed of about 5000 lb/hr, the material is conveyed at a rate of about 143 ft3/hr, if the material is ¼″ thick and is in a trough 5.5 ft wide, the material is conveyed at a rate of about 1250 ft/hr (about 21 ft/min) Rates of conveying the material can therefore vary greatly. Preferably, for example, a ¼″ thick layer of biomass, is conveyed at speeds of between about 5 and 100 ft/min (e.g. between about 5 and 100 ft/min, between about 6 and 100 ft/min, between about 7 and 100 ft/min, between about 8 and 100 ft/min, between about 9 and 100 ft/min, between about 10 and 100 ft/min, between about 11 and 100 ft/min, between about 12 and 100 ft/min, between about 13 and 100 ft/min, between about 14 and 100 ft/min, between about 15 and 100 ft/min, between about 20 and 100 ft/min, between about 30 and 100 ft/min, between about 40 and 100 ft/min, between about 2 and 60 ft/min, between about 3 and 60 ft/min, between about 5 and 60 ft/min, between about 6 and 60 ft/min, between about 7 and 60 ft/min, between about 8 and 60 ft/min, between about 9 and 60 ft/min, between about 10 and 60 ft/min, between about 15 and 60 ft/min, between about 20 and 60 ft/min, between about 30 and 60 ft/min, between about 40 and 60 ft/min, between about 2 and 50 ft/min, between about 3 and 50 ft/min, between about 5 and 50 ft/min, between about 6 and 50 ft/min, between about 7 and 50 ft/min, between about 8 and 50 ft/min, between about 9 and 50 ft/min, between about 10 and 50 ft/min, between about 15 and 50 ft/min, between about 20 and 50 ft/min, between about 30 and 50 ft/min, between about 40 and 50 ft/min) It is preferable that the material be conveyed at a constant rate, for example, to help maintain a constant irradiation of the material as it passes under the electron beam (e.g., shower, field).

The vibratory conveyors described can include screens used for sieving and sorting materials. Port openings on the side or bottom of the troughs can be used for sorting, selecting or removing specific materials, for example, by size or shape. Some conveyors have counterbalances to reduce the dynamic forces on the support structure. Some vibratory conveyors are configured as spiral elevators, are designed to curve around surfaces and/or are designed to drop material from one conveyor to another (e.g., in a step, cascade or as a series of steps or a stair). Along with conveying materials, conveyors can be used by themselves or coupled with other equipment or systems, for screening, separating, sorting, classifying, distributing, sizing, inspection, picking, metal removing, freezing, blending, mixing, orienting, heating, cooking, drying, dewatering, cleaning, washing, leaching, quenching, coating, de-dusting and/or feeding. The conveyors can also include covers (e.g., dust-tight covers), side discharge gates, bottom discharge gates, special liners (e.g., anti-stick, stainless steel, rubber, custom steal, and or grooved), divided troughs, quench pools, screens, perforated plates, detectors (e.g., metal detectors), high temperature designs, food grade designs, heaters, dryers and or coolers. In addition, the trough can be of various shapes, for example, flat bottomed, vee shaped bottom, flanged at the top, curved bottom, flat with ridges in any direction, tubular, half pipe, covered or any combinations of these. In particular, the conveyors can be coupled with an irradiation systems and/or equipment.

The conveyors (e.g., vibratory conveyor) can be made of corrosion resistant materials. The conveyors can utilize structural materials that include stainless steel (e.g., 304, 316 stainless steel, HASTELLOY® ALLOYS and INCONEL® Alloys). For example, HASTELLOY® Corrosion-Resistant alloys from Hynes (Kokomo, Ind., USA) such as HASTELLOY® B-3® ALLOY, HASTELLOY® HYBRID-BC1® ALLOY, HASTELLOY® C-4 ALLOY, HASTELLOY® C-22® ALLOY, HASTELLOY® C-22HS® ALLOY, HASTELLOY® C-276 ALLOY, HASTELLOY® C-2000® ALLOY, HASTELLOY® G-30® ALLOY, HASTELLOY® G-35® ALLOY, HASTELLOY® N ALLOY and HASTELLOY® ULTIMET® alloy.

The vibratory conveyors can include non-stick release coatings, for example, TUFFLON™ (Dupont, Del., USA). The vibratory conveyors can also include corrosion resistant coatings. For example, coatings that can be supplied from Metal Coatings Corp (Houston, Tex., USA) and others such as Fluoropolymer, XYLAN®, Molybdenum Disulfide, Epoxy Phenolic, Phosphate-ferrous metal coating, Polyurethane-high gloss topcoat for epoxy, inorganic zinc, Poly Tetrafluoro ethylene, PPS/RYTON®, fluorinated ethylene propylene, PVDF/DYKOR®, ECTFE/HALAR® and Ceramic Epoxy Coating. The coatings can improve resistance to process gases (e.g., ozone), chemical corrosion, pitting corrosion, galling corrosion and oxidation.

Optionally, in addition to the conveying systems described herein, one or more other conveying systems can be enclosed. When using an enclosure, the enclosed conveyor can also be purged with an inert gas so as to maintain an atmosphere at a reduced oxygen level. Keeping oxygen levels low avoids the formation of ozone which in some instances is undesirable due to its reactive and toxic nature. For example, the oxygen can be less than about 20% (e.g., less than about 10%, less than about 1%, less than about 0.1%, less than about 0.01%, or even less than about 0.001% oxygen). Purging can be done with an inert gas including, but not limited to, nitrogen, argon, helium or carbon dioxide. This can be supplied, for example, from a boil off of a liquid source (e.g., liquid nitrogen or helium), generated or separated from air in situ, or supplied from tanks. The inert gas can be recirculated and any residual oxygen can be removed using a catalyst, such as a copper catalyst bed. Alternatively, combinations of purging, recirculating and oxygen removal can be done to keep the oxygen levels low.

The enclosed conveyor can also be purged with a reactive gas that can react with the biomass. This can be done before, during or after the irradiation process. The reactive gas can be, but is not limited to, nitrous oxide, ammonia, oxygen, ozone, hydrocarbons, aromatic compounds, amides, peroxides, azides, halides, oxyhalides, phosphides, phosphines, arsines, sulfides, thiols, boranes and/or hydrides. The reactive gas can be activated in the enclosure, e.g., by irradiation (e.g., electron beam, UV irradiation, microwave irradiation, heating, IR radiation), so that it reacts with the biomass. The biomass itself can be activated, for example, by irradiation. Preferably the biomass is activated by the electron beam, to produce radicals which then react with the activated or unactivated reactive gas, e.g., by radical coupling or quenching.

Purging gases supplied to an enclosed conveyor can also be cooled, for example, below about 25° C., below about 0° C., below about −40° C., below about −80° C., below about −120° C. For example, the gas can be boiled off from a compressed gas such as liquid nitrogen or sublimed from solid carbon dioxide. As an alternative example, the gas can be cooled by a chiller or part of or the entire conveyor can be cooled.

### Other Embodiments

Any material, processes or processed materials discussed herein can be used to make products and/or intermediates such as composites, fillers, binders, plastic additives, adsorbents and controlled release agents. The methods can include densification, for example, by applying pressure and heat to the materials. For example, composites can be made by combining fibrous materials with a resin or polymer. For example, radiation cross-linkable resin, e.g., a thermoplastic resin can be combined with a fibrous material to provide a fibrous material/cross-linkable resin combination. Such materials can be, for example, useful as building materials, protective sheets, containers and other structural materials (e.g., molded and/or extruded products). Absorbents can be, for example, in the form of pellets, chips, fibers and/or sheets. Adsorbents can be used, for example, as pet bedding, packaging material or in pollution control systems. Controlled release matrices can also be the form of, for example, pellets, chips, fibers and or sheets. The controlled release matrices can, for example, be used to release drugs, biocides, fragrances. For example, composites, absorbents and control release agents and their uses are described in International Serial No. PCT/US2006/010648, filed Mar. 23, 2006, and U.S. Pat. No. 8,074,910 filed Nov. 22, 2011, the entire disclosures of which are herein incorporated by reference.

In some instances the biomass material is treated at a first level to reduce recalcitrance, e.g., utilizing accelerated electrons, to selectively release one or more sugars (e.g., xylose). The biomass can then be treated to a second level to release one or more other sugars (e.g., glucose). Optionally, the biomass can be dried between treatments. The treatments can include applying chemical and biochemical treatments to release the sugars. For example, a biomass material can be treated to a level of less than about 20 Mrad (e.g., less than about 15 Mrad, less than about 10 Mrad, less than about 5 Mrad, less than about 2 Mrad) and then treated with a solution of sulfuric acid, containing less than 10% sulfuric acid (e.g., less than about 9%, less than about 8%, less than about 7%, less than about 6%, less than about 5%, less than about 4%, less than about 3%, less than about 2%, less than about 1%, less than about 0.75%, less than about 0.50%, less than about 0.25%) to release xylose. Xylose, for example, that is released into solution, can be separated from solids and optionally the solids washed with a solvent/solution (e.g., with water and/or acidified water). Optionally, the solids can be dried, for example, in air and/or under vacuum optionally with heating (e.g., below about 150 deg C, below about 120 deg C) to a water content below about 25 wt % (below about 20 wt. %, below about 15 wt. %, below about 10 wt. %, below about 5 wt. %). The solids can then be treated with a level of less than about 30 Mrad (e.g., less than about 25 Mrad, less than about 20 Mrad, less than about 15 Mrad, less than about 10 Mrad, less than about 5 Mrad, less than about 1 Mrad or even not at all) and then treated with an enzyme (e.g., a cellulase) to release glucose. The glucose (e.g., glucose in solution) can be separated from the remaining solids. The solids can then be further processed, for example, utilized to make energy or other products (e.g., lignin derived products).

### Flavors, Fragrances and Colorants

Any of the products and/or intermediates described herein, for example, produced by the processes, systems and/or equipment described herein, can be combined with flavors, fragrances, colorants and/or mixtures of these. For example, any one or more of (optionally along with flavors, fragrances and/or colorants) sugars, organic acids, fuels, polyols, such as sugar alcohols, biomass, fibers and composites can be combined with (e.g., formulated, mixed or reacted) or used to make other products. For example, one or more such product can be used to make soaps, detergents, candies, drinks (e.g., cola, wine, beer, liquors such as gin or vodka, sports drinks, coffees, teas), pharmaceuticals, adhesives, sheets (e.g., woven, none woven, filters, tissues) and/or composites (e.g., boards). For example, one or more such product can be combined with herbs, flowers, petals, spices, vitamins, potpourri, or candles. For example, the formulated, mixed or reacted combinations can have flavors/fragrances of grapefruit, orange, apple, raspberry, banana, lettuce, celery, cinnamon, chocolate, vanilla, peppermint, mint, onion, garlic, pepper, saffron, ginger, milk, wine, beer, tea, lean beef, fish, clams, olive oil, coconut fat, pork fat, butter fat, beef bouillon, legume, potatoes, marmalade, ham, coffee and cheeses.

Flavors, fragrances and colorants can be added in any amount, such as between about 0.001 wt. % to about 30 wt. %, e.g., between about 0.01 to about 20, between about 0.05 to about 10, or between about 0.1 wt. % to about 5 wt. %. These can be formulated, mixed and or reacted (e.g., with any one of more product or intermediate described herein) by any means and in any order or sequence (e.g., agitated, mixed, emulsified, gelled, infused, heated, sonicated, and/or suspended). Fillers, binders, emulsifier, antioxidants can also be utilized, for example, protein gels, starches and silica.

In one embodiment the flavors, fragrances and colorants can be added to the biomass immediately after the biomass is irradiated such that the reactive sites created by the irradiation may react with reactive compatible sites of the flavors, fragrances, and colorants.

The flavors, fragrances and colorants can be natural and/or synthetic materials. These materials can be one or more of a compound, a composition or mixtures of these (e.g., a formulated or natural composition of several compounds). Optionally the flavors, fragrances, antioxidants and colorants can be derived biologically, for example, from a fermentation process (e.g., fermentation of saccharified materials as described herein). Alternatively, or additionally these flavors, fragrances and colorants can be harvested from a whole organism (e.g., plant, fungus, animal, bacteria or yeast) or a part of an organism. The organism can be collected and or extracted to provide color, flavors, fragrances and/or antioxidant by any means including utilizing the methods, systems and equipment described herein, hot water extraction, supercritical fluid extraction, chemical extraction (e.g., solvent or reactive extraction including acids and bases), mechanical extraction (e.g., pressing, comminuting, filtering), utilizing an enzyme, utilizing a bacteria such as to break down a starting material, and combinations of these methods. The compounds can be derived by a chemical reaction, for example, the combination of a sugar (e.g., as produced as described herein) with an amino acid (Maillard reaction). The flavor, fragrance, antioxidant and/or colorant can be an intermediate and or product produced by the methods, equipment or systems described herein, for example, and ester and a lignin derived product.

Some examples of flavor, fragrances or colorants are polyphenols. Polyphenols are pigments responsible for the red, purple and blue colorants of many fruits, vegetables, cereal grains, and flowers. Polyphenols also can have antioxidant properties and often have a bitter taste. The antioxidant properties make these important preservatives. On class of polyphenols are the flavonoids, such as Anthocyanidines, flavanonols, flavan-3-ols, s, flavanones and flavanonols. Other phenolic compounds that can be used include phenolic acids and their esters, such as chlorogenic acid and polymeric tannins.

Among the colorants inorganic compounds, minerals or organic compounds can be used, for example, titanium dioxide, zinc oxide, aluminum oxide, cadmium yellow (E.g., CdS), cadmium orange (e.g., CdS with some Se), alizarin crimson (e.g., synthetic or non-synthetic rose madder), ultramarine (e.g., synthetic ultramarine, natural ultramarine, synthetic ultramarine violet), cobalt blue, cobalt yellow, cobalt green, viridian (e.g., hydrated chromium(III)oxide), chalcophylite, conichalcite, cornubite, cornwallite and liroconite. Black pigments such as carbon black and self-dispersed blacks may be used.

Some flavors and fragrances that can be utilized include ACALEA TBHQ, ACET C-6, ALLYL AMYL GLYCOLATE, ALPHA TERPINEOL, AMBRETTOLIDE, AMBRINOL 95, ANDRANE, APHERMATE, APPLELIDE, BACDANOL®, BERGAMAL, BETA IONONE EPDXIDE, BETA NAPHTHYL ISO-BUTYL ETHER, BICYCLONONALACTONE, BORNAFIX®, CANTHOXAL, CASHMERAN®, CASHMERAN® VELVET, CASSIFFIX®, CEDRAFIX, CEDRAMBER®, CEDRYL ACETATE, CELESTOLIDE, CINNAMALVA, CITRAL DIMETHYL ACETATE, CITROLATE™, CITRONELLOL 700, CITRONELLOL 950, CITRONELLOL COEUR, CITRONELLYL ACETATE, CITRONELLYL ACETATE PURE, CITRONELLYL FORMATE, CLARYCET, CLONAL, CONIFERAN, CONIFERAN PURE, CORTEX ALDEHYDE 50% PEOMOSA, CYCLABUTE, CYCLACET®, CYCLAPROP®, CYCLEMAX™, CYCLOHEXYL ETHYL ACETATE, DAMASCOL, DELTA DAMASCONE, DIHYDRO CYCLACET, DIHYDRO MYRCENOL, DIHYDRO TERPINEOL, DIHYDRO TERPINYL ACETATE, DIMETHYL CYCLORMOL, DIMETHYL OCTANOL PQ, DIMYRCETOL, DIOLA, DIPENTENE, DULCINYL® RECRYSTALLIZED, ETHYL-3-PHENYL GLYCIDATE, FLEURAMONE, FLEURANIL, FLORAL SUPER, FLORALOZONE, FLORIFFOL, FRAISTONE, FRUCTONE, GALAXOLIDE® 50, GALAXOLIDE® 50 BB, GALAXOLIDE® 50 IPM, GALAXOLIDE® UNDILUTED, GALBASCONE, GERALDEHYDE, GERANIOL 5020, GERANIOL 600 TYPE, GERANIOL 950, GERANIOL 980 (PURE), GERANIOL CFT COEUR, GERANIOL COEUR, GERANYL ACETATE COEUR, GERANYL ACETATE, PURE, GERANYL FORMATE, GRISALVA, GUAIYL ACETATE, HELIONAL™, HERBAC, HERBALIME™, HEXADECANOLIDE, HEXALON, HEXENYL SALICYLATE CIS 3-, HYACINTH BODY, HYACINTH BODY NO. 3, HYDRATROPIC ALDEHYDE.DMA, HYDROXYOL, INDOLAROME, INTRELEVEN ALDEHYDE, INTRELEVEN ALDEHYDE SPECIAL, IONONE ALPHA, IONONE BETA, ISO CYCLO CITRAL, ISO CYCLO GERANIOL, ISO E SUPER®, ISOBUTYL QUINOLINE, JASMAL, JESSEMAL®, KHARISMAL®, KHARISMAL® SUPER, KHUSINIL, KOAVONE®, KOHINOOL®, LIFFAROME™, LIMOXAL, LINDENOL™, LYRAL®, LYRAME SUPER, MANDARIN ALD 10% TRI ETH, CITR, MARITIMA, MCK CHINESE, MEIJIFN™, MELAFLEUR, MELOZONE, METHYL ANTHRANILATE, METHYL IONONE ALPHA EXTRA, METHYL IONONE GAMMA A, METHYL IONONE GAMMA COEUR, METHYL IONONE GAMMA PURE, METHYL LAVENDER KETONE, MONTAVERDI®, MUGUESIA, MUGUET ALDEHYDE 50, MUSK Z4, MYRAC ALDEHYDE, MYRCENYL ACETATE, NECTARATE™, NEROL 900, NERYL ACETATE, OCIMENE, OCTACETAL, ORANGE FLOWER ETHER, ORIVONE, ORRINIFF 25%, OXASPIRANE, OZOFLEUR, PAMPLEFLEUR®, PEOMOSA, PHENOXANOL®, PICONIA, PRECYCLEMONE B, PRENYL ACETATE, PRISMANTOL, RESEDA BODY, ROSALVA, ROSAMUSK, SANJINOL, SANTALIFF™, SYVERTAL, TERPINEOL, TERPINOLENE 20, TERPINOLENE 90 PQ, TERPINOLENE RECT., TERPINYL ACETATE, TERPINYL ACETATE JAX, TETRAHYDRO, MUGUOL®, TETRAHYDRO MYRCENOL, TETRAMERAN, TIMBERSILK™, TOBACAROL, TRIMOFIX® O TT, TRIPLAL®, TRISAMBER®, VANORIS, VERDOX™ VERDOX™ HC, VERTENEX®, VERTENEX® HC, VERTOFIX® COEUR, VERTOLIFF, VERTOLIFF ISO, VIOLIFF, VIVALDIE, ZENOLIDE, ABS INDIA 75 PCT MIGLYOL, ABS MOROCCO 50 PCT DPG, ABS MOROCCO 50 PCT TEC, ABSOLUTE FRENCH, ABSOLUTE INDIA, ABSOLUTE MD 50 PCT BB, ABSOLUTE MOROCCO, CONCENTRATE PG, TINCTURE 20 PCT, AMBERGRIS, AMBRETTE ABSOLUTE, AMBRETTE SEED OIL, ARMOISE OIL 70 PCT THUYONE, BASIL ABSOLUTE GRAND VERT, BASIL GRAND VERT ABS MD, BASIL OIL GRAND VERT, BASIL OIL VERVEINA, BASIL OIL VIETNAM, BAY OIL TERPENELESS, BEESWAX ABS N G, BEESWAX ABSOLUTE, BENZOIN RESINOID SIAM, BENZOIN RESINOID SIAM 50 PCT DPG, BENZOIN RESINOID SIAM 50 PCT PG, BENZOIN RESINOID SIAM 70.5 PCT TEC, BLACKCURRANT BUD ABS 65 PCT PG, BLACKCURRANT BUD ABS MD 37 PCT TEC, BLACKCURRANT BUD ABS MIGLYOL, BLACKCURRANT BUD ABSOLUTE BURGUNDY, BOIS DE ROSE OIL, BRAN ABSOLUTE, BRAN RESINOID, BROOM ABSOLUTE ITALY, CARDAMOM GUATEMALA CO2 EXTRACT, CARDAMOM OIL GUATEMALA, CARDAMOM OIL INDIA, CARROT HEART, CASSIE ABSOLUTE EGYPT, CASSIE ABSOLUTE MD 50 PCT IPM, CASTOREUM ABS 90 PCT TEC, CASTOREUM ABS C 50 PCT MIGLYOL, CASTOREUM ABSOLUTE, CASTOREUM RESINOID, CASTOREUM RESINOID 50 PCT DPG, CEDROL CEDRENE, CEDRUS ATLANTICA OIL REDIST, CHAMOMILE OIL ROMAN, CHAMOMILE OIL WILD, CHAMOMILE OIL WILD LOW LIMONENE, CINNAMON BARK OIL CEYLAN, CISTE ABSOLUTE, CISTE ABSOLUTE COLORLESS, CITRONELLA OIL ASIA IRON FREE, CIVET ABS 75 PCT PG, CIVET ABSOLUTE, CIVET TINCTURE 10 PCT, CLARY SAGE ABS FRENCH DECOL, CLARY SAGE ABSOLUTE FRENCH, CLARY SAGE C′LESS 50 PCT PG, CLARY SAGE OIL FRENCH, COPAIBA BALSAM, COPAIBA BALSAM OIL, CORIANDER SEED OIL, CYPRESS OIL, CYPRESS OIL ORGANIC, DAVANA OIL, GALBANOL, GALBANUM ABSOLUTE COLORLESS, GALBANUM OIL, GALBANUM RESINOID, GALBANUM RESINOID 50 PCT DPG, GALBANUM RESINOID HERCOLYN BHT, GALBANUM RESINOID TEC BHT, GENTIANE ABSOLUTE MD 20 PCT BB, GENTIANE CONCRETE, GERANIUM ABS EGYPT MD, GERANIUM ABSOLUTE EGYPT, GERANIUM OIL CHINA, GERANIUM OIL EGYPT, GINGER OIL 624, GINGER OIL RECTIFIED SOLUBLE, GUAIACWOOD HEART, HAY ABS MD 50 PCT BB, HAY ABSOLUTE, HAY ABSOLUTE MD 50 PCT TEC, HEALINGWOOD, HYSSOP OIL ORGANIC, IMMORTELLE ABS YUGO MD 50 PCT TEC, IMMORTELLE ABSOLUTE SPAIN, IMMORTELLE ABSOLUTE YUGO, JASMIN ABS INDIA MD, JASMIN ABSOLUTE EGYPT, JASMIN ABSOLUTE INDIA, ASMIN ABSOLUTE MOROCCO, JASMIN ABSOLUTE SAMBAC, JONQUILLE ABS MD 20 PCT BB, JONQUILLE ABSOLUTE France, JUNIPER BERRY OIL FLG, JUNIPER BERRY OIL RECTIFIED SOLUBLE, LABDANUM RESINOID 50 PCT TEC, LABDANUM RESINOID BB, LABDANUM RESINOID MD, LABDANUM RESINOID MD 50 PCT BB, LAVANDIN ABSOLUTE H, LAVANDIN ABSOLUTE MD, LAVANDIN OIL ABRIAL ORGANIC, LAVANDIN OIL GROSSO ORGANIC, LAVANDIN OIL SUPER, LAVENDER ABSOLUTE H, LAVENDER ABSOLUTE MD, LAVENDER OIL COUMARIN FREE, LAVENDER OIL COUMARIN FREE ORGANIC, LAVENDER OIL MAILLETTE ORGANIC, LAVENDER OIL MT, MACE ABSOLUTE BB, MAGNOLIA FLOWER OIL LOW METHYL EUGENOL, MAGNOLIA FLOWER OIL, MAGNOLIA FLOWER OIL MD, MAGNOLIA LEAF OIL, MANDARIN OIL MD, MANDARIN OIL MD BHT, MATE ABSOLUTE BB, MOSS TREE ABSOLUTE MD TEX IFRA 43, MOSS-OAK ABS MD TEC IFRA 43, MOSS-OAK ABSOLUTE IFRA 43, MOSS-TREE ABSOLUTE MD IPM IFRA 43, MYRRH RESINOID BB, MYRRH RESINOID MD, MYRRH RESINOID TEC, MYRTLE OIL IRON FREE, MYRTLE OIL TUNISIA RECTIFIED, NARCISSE ABS MD 20 PCT BB, NARCISSE ABSOLUTE FRENCH, NEROLI OIL TUNISIA, NUTMEG OIL TERPENELESS, OEILLET ABSOLUTE, OLIBANUM RESINOID, OLIBANUM RESINOID BB, OLIBANUM RESINOID DPG, OLIBANUM RESINOID EXTRA 50 PCT DPG, OLIBANUM RESINOID MD, OLIBANUM RESINOID MD 50 PCT DPG, OLIBANUM RESINOID TEC, OPOPONAX RESINOID TEC, ORANGE BIGARADE OIL MD BHT, ORANGE BIGARADE OIL MD SCFC, ORANGE FLOWER ABSOLUTE TUNISIA, ORANGE FLOWER WATER ABSOLUTE TUNISIA, ORANGE LEAF ABSOLUTE, ORANGE LEAF WATER ABSOLUTE TUNISIA, ORRIS ABSOLUTE ITALY, ORRIS CONCRETE 15 PCT IRONE, ORRIS CONCRETE 8 PCT IRONE, ORRIS NATURAL 15 PCT IRONE 4095C, ORRIS NATURAL 8 PCT IRONE 2942C, ORRIS RESINOID, OSMANTHUS ABSOLUTE, OSMANTHUS ABSOLUTE MD 50 PCT BB, PATCHOULI HEART N^{o}3, PATCHOULI OIL INDONESIA, PATCHOULI OIL INDONESIA IRON FREE, PATCHOULI OIL INDONESIA MD, PATCHOULI OIL REDIST, PENNYROYAL HEART, PEPPERMINT ABSOLUTE MD, PETITGRAIN BIGARADE OIL TUNISIA, PETITGRAIN CITRONNIER OIL, PETITGRAIN OIL PARAGUAY TERPENELESS, PETITGRAIN OIL TERPENELESS STAB, PIMENTO BERRY OIL, PIMENTO LEAF OIL, RHODINOL EX GERANIUM CHINA, ROSE ABS BULGARIAN LOW METHYL EUGENOL, ROSE ABS MOROCCO LOW METHYL EUGENOL, ROSE ABS TURKISH LOW METHYL EUGENOL, ROSE ABSOLUTE, ROSE ABSOLUTE BULGARIAN, ROSE ABSOLUTE DAMASCENA, ROSE ABSOLUTE MD, ROSE ABSOLUTE MOROCCO, ROSE ABSOLUTE TURKISH, ROSE OIL BULGARIAN, ROSE OIL DAMASCENA LOW METHYL EUGENOL, ROSE OIL TURKISH, ROSEMARY OIL CAMPHOR ORGANIC, ROSEMARY OIL TUNISIA, SANDALWOOD OIL INDIA, SANDALWOOD OIL INDIA RECTIFIED, SANTALOL, SCHINUS MOLLE OIL, ST JOHN BREAD TINCTURE 10 PCT, STYRAX RESINOID, STYRAX RESINOID, TAGETE OIL, TEA TREE HEART, TONKA BEAN ABS 50 PCT SOLVENTS, TONKA BEAN ABSOLUTE, TUBEROSE ABSOLUTE INDIA, VETIVER HEART EXTRA, VETIVER OIL HAITI, VETIVER OIL HAITI MD, VETIVER OIL JAVA, VETIVER OIL JAVA MD, VIOLET LEAF ABSOLUTE EGYPT, VIOLET LEAF ABSOLUTE EGYPT DECOL, VIOLET LEAF ABSOLUTE FRENCH, VIOLET LEAF ABSOLUTE MD 50 PCT BB, WORMWOOD OIL TERPENELESS, YLANG EXTRA OIL, YLANG III OIL and combinations of these.

The colorants can be among those listed in the Color Index International by the Society of Dyers and Colourists. Colorants include dyes and pigments and include those commonly used for coloring textiles, paints, inks and inkjet inks. Some colorants that can be utilized include carotenoids, arylide yellows, diarylide yellows, β-naphthols, naphthols, benzimidazolones, disazo condensation pigments, pyrazolones, nickel azo yellow, phthalocyanines, quinacridones, perylenes and perinones, isoindolinone and isoindoline pigments, triarylcarbonium pigments, diketopyrrolo-pyrrole pigments, thioindigoids. Cartenoids include, for example, alpha-carotene, beta-carotene, gamma-carotene, lycopene, lutein and astaxanthin, Annatto extract, Dehydrated beets (beet powder), Canthaxanthin, Caramel, β-Apo-8′-carotenal, Cochineal extract, Carmine, Sodium copper chlorophyllin, Toasted partially defatted cooked cottonseed flour, Ferrous gluconate, Ferrous lactate, Grape color extract, Grape skin extract (enocianina), Carrot oil, Paprika, Paprika oleoresin, Mica-based pearlescent pigments, Riboflavin, Saffron, Titanium dioxide, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Potassium sodium copper chlorophyllin (chlorophyllin-copper complex), Dihydroxyacetone, Bismuth oxychloride, Ferric ammonium ferrocyanide, Ferric ferrocyanide, Chromium hydroxide green, Chromium oxide greens, Guanine, Pyrophyllite, Talc, Aluminum powder, Bronze powder, Copper powder, Zinc oxide, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3 (3), D&C Brown No. 1, Ext. D&C, Chromium-cobalt-aluminum oxide, Ferric ammonium citrate, Pyrogallol, Logwood extract, 1,4-Bis[(2-hydroxy-ethyl)amino]-9,10-anthracenedione bis(2-propenoic)ester copolymers, 1,4-Bis[(2-methylphenyl)amino]-9,10-anthracenedione, 1,4-Bis[4-(2-methacryloxyethyl)phenylamino]anthraquinone copolymers, Carbazole violet, Chlorophyllin-copper complex, Chromium-cobalt-aluminum oxide, C.I. Vat Orange 1, 2-[[2,5-Diethoxy-4-[(4-methylphenyl)thiol]phenyl]azo]-1,3,5-benzenetriol, 16,23-Dihydrodinaphtho[2,3-a:2′,3′-i]naphth[2′,3′:6,7]indolo[2,3-c]carbazole-5,10,15,17,22,24-hexone, N,N′-(9,10-Dihydro-9,10-dioxo-1,5-anthracenediyl) bisbenzamide, 7,16-Dichloro-6,15-dihydro-5,9,14,18-anthrazinetetrone, 16,17-Dimethoxydinaphtho (1,2,3-cd:3′,2′,1′-lm) perylene-5,10-dione, Poly(hydroxyethyl methacrylate)-dye copolymers(3), Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-2-phenyl-3H-pyrazol-3-one (solvent Yellow 18), 6-Ethoxy-2-(6-ethoxy-3-oxobenzo[b]thien-2(3H)-ylidene) benzo[b]thiophen-3(2H)-one, Phthalocyanine green, Vinyl alcohol/methyl methacrylate-dye reaction products, C.I. Reactive Red 180, C.I. Reactive Black 5, C.I. Reactive Orange 78, C.I. Reactive Yellow 15, C.I. Reactive Blue 21, Disodium 1-amino-4-[[4-[(2-bromo-1-oxoally)amino]-2-sulphonatophenyl]amino]-9,10-dihydro-9,10-dioxoanthracene-2-sulphonate (Reactive Blue 69), D&C Blue No. 9, [Phthalocyaninato(2-)] copper and mixtures of these.

### Examples

### Succinic Acid Production from Saccharified Corncob Using Actinobacillus succinogenes

Material and Methods

Succinic Acid Producing Strains Tested:

Actinobacillus succinogenes

Anaerobiospirillum succiniciproducens

Mannheimia succiniciproducens

Recombinant Escherichia coli: Aerobic, PEP carboxylase over-expressing E. coli

Seed Culture

Cells from a frozen (−80° C.) cell bank were cultivated in propagation medium (BD Trypticase Soy media) at 30° C., with 150 rpm stirring for 20 hours. This seed culture was transferred to a 1.2 L bioreactors charged with media as describe below. Trypticase Soy (TS) includes 17 g/L Pancreatic digest of casein, 3 g/L papaic digest of soybean meal, 2.5 g/L glucose, 5 g/L sodium chloride and 2.5 g/L dipotassium phosphate.

Media and Conditions Testing

Table 1 outlines experiments to determine the impact of various media components and conditions on succinic acid production.

TABLE 1
Succinic acid production with Actinobacillus succinogenes

| | | | | Range— |
|---|---|---|---|---|
| | | | Range— | Currently |
| Media | | Range— | Currently | Most |
| Component | Test Parameter | Tested | preferred^{a} | Preferred^{b} |
| Initial sugar | Succinic acid | 40-100 g/L | 40-100 g/L | 50-60 g/L |
| concentration | concentration | | | |
| Nitrogen | Succinic acid | Yeast extract, | Yeast extract, | Yeast extract |
| Sources | concentration | Trypticase, | Tryptone, | |
| Tested | | peptone, | Trypticase | |
| | | Corn steep, | | |
| | | Tryptone, | | |
| | | Peptone | | |
| Yeast | Succinic acid | 0-20 g/L | 10-20 g/L | 20 g/L |
| Extract^{a} | concentration | | | |
| Magnesium | Succinic acid | 0-8 | 3-5% | 3-5% |
| carbonate | concentration | wt. %/vol. % | wt.%/vol.% | wt. %/vol. % |
| Inorganic | Succinic acid | With or | With | With |
| salts^{b} | concentration | without | | |
| | | | | Range— |
| Physical | | Range— | Range— | Most |
| Condition | Test Parameter | Tested | preferred | Preferred |
| Temperature | Succinic acid | 28-48 deg C | 34-40 deg C | 37-40 deg C |
| | concentration | | | |
| Agitation | Succinic acid | 50-500 rpm | 50-500 rpm | 50-500 rpm |
| (in 1.2 L | concentration | | | |
| reactor) | | | | |
| CO₂ gas | Succinic acid | 0-0.5 VVM | 0-0.5 VVM | 0 VVM |
| sparging | concentration | | | |

^{a}Fluka brand yeast extract was used.
^{b}NaH₂PO₄ (1.5 g/L), Na₂HPO₄ (1.5 g), NaCl (1.0 g/L), MgCl₂ (0.2 g/L), CaCl₂ (0.2 g/L).
The pH was regulated by addition of MgCO₃.
Media is also supplemented with CO₂ gas through a sparge tub.

Based on the results on the media component and conditions tests, the following media and conditions were used for subsequent testing. A 0.7 L of culture volume in 1.2 L vessel bioreactor (New Brunswick) was tested. 1% of 20-hour-cultured seed was used for inoculation. No CO₂ sparging was used. Magnesum carbonate was added to 5% (w/v) to maintain pH between 5 and 6. The temperature was maintained at 37° C. Antifoam 204 was added (0.1%, 1 ml/L) at the beginning of the culture and then was not added any more. Inorganic salts were used.

Results

1. Bioreactor Culture of Actinobacillus succinogenes (ATCC 55618) with Reagent Glucose and Xylose.

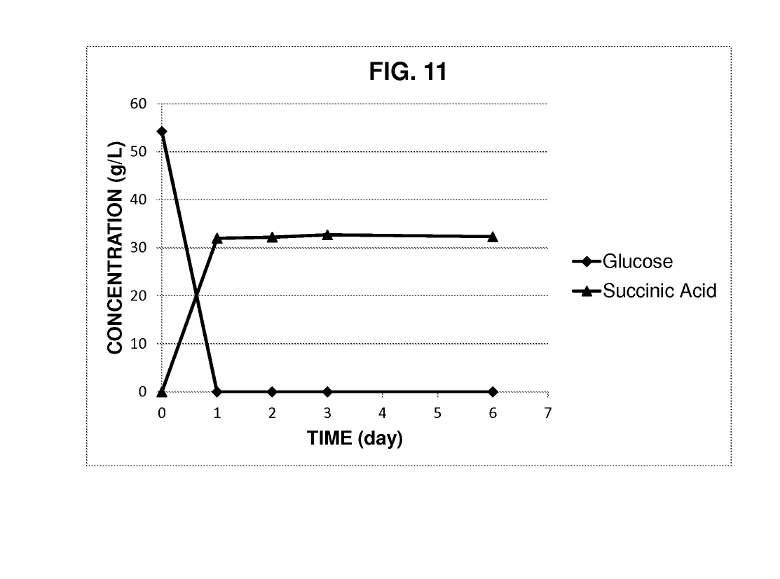

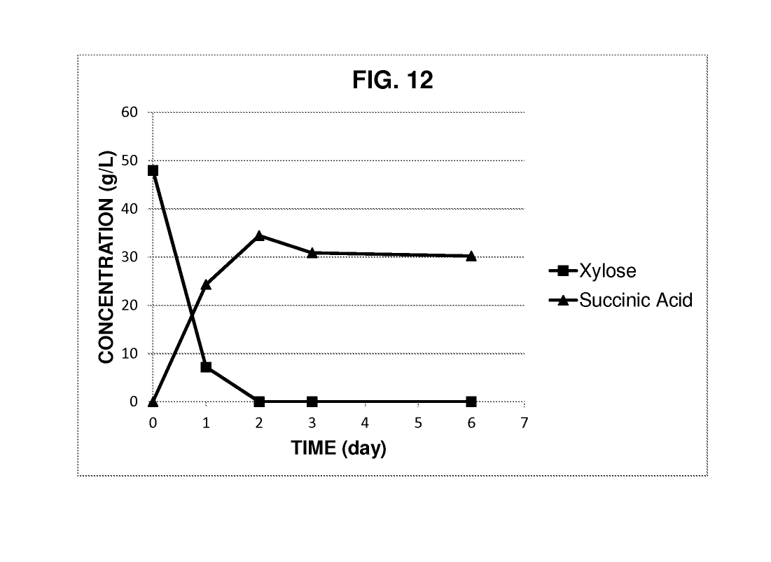

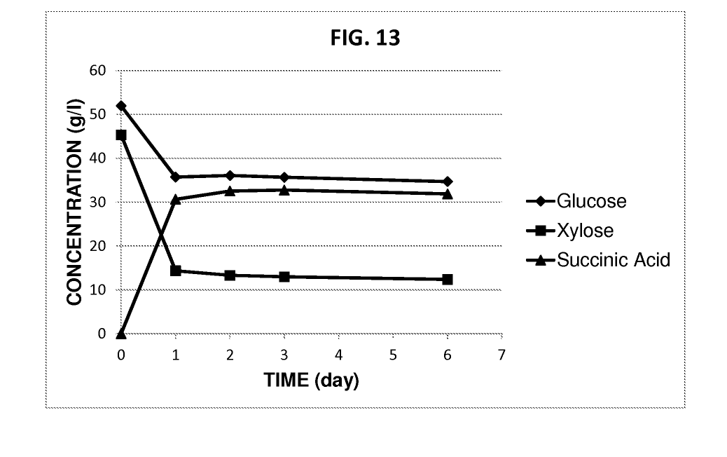

Three independent experiments using the above media and conditions were conducted. The first used glucose as the carbon source. FIG. 11 is a plot showing the consumption of glucose and production of succinic acid in this first experiment. A second experiment used xylose as the carbon source. FIG. 12 is a plot showing the consumption of xylose and production of succinic acid for this second experiment. A third experiment used both glucose and xylose as the carbon source. FIG. 13 is a plot showing the consumption of glucose+xylose and production of succinic acid in the third experiment.

2. Bioreactor Culture of Actinobacillus succinogenes (ATCC 55618) with Saccharified Corncob.

In addition to the media components described above (most preferred), sugar solution from saccharified biomass was used. The sugar solution included saccharified corncob that had been hammer milled and irradiated with about 35 Mrad of electron beam irradiation. For example, saccharified corn cob can be prepared as described in U.S. provisional application Ser. No. 61/774,723 filed on Mar. 8, 2013 the entire disclosure of which is herein incorporated by reference.

Actinobacillus succinogenes (ATCC 55618) was cultured in the production medium in the 1.2 L bioreactor (culture volume volume is 0.7 L) with various medium components conditions (table 1). Culture period was 3 to 5 days.

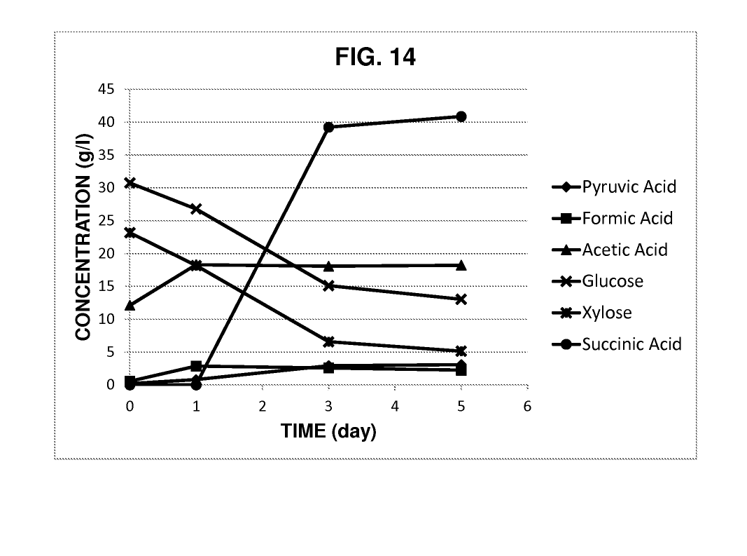

FIG. 14 is a plot of sugars consumed and products produced using a 1.2 L Bioreactor culture of Actinobacillus succinogenes (ATCC 55618) with the preferred conditions and saccharified corn cob. The conditions were: media components Saccharified corncob, 20 g/L yeast extract, inorganic salts; and physical conditions: 37° C. and 200 rpm.

Simultaneous consumption of glucose and xylose was observed, where glucose started near 30 g/L and decreased to about 12 g/L and Xylose started at about 24 g/L and decreased to about 5 g/L. Cellobiose was not consumed (not shown). About 30 g/L of succinic acid was produced so the yield based on glucose and xylose was just over about 50%.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to most preferred embodiments thereof, it will be understood by those skilled in the art that various changes in form and details may be made therein without departing from the scope of the invention encompassed by the appended claims.

1. A method for making a product comprising:
treating a reduced recalcitrance lignocellulosic or cellulosic material with one or more enzymes and/or organisms to produce a poly carboxylic acid; and
converting the poly carboxylic acid to the product.

2. The method of claim 1, wherein a feedstock is pretreated with at least one of irradiation, sonication, oxidation, pyrolysis and steam explosion to produce the reduced recalcitrance lignocellulosic or cellulosic material.

3. The method of claim 2, wherein irradiation is performed with an electron beam.

4. The method of claim 1, wherein converting the poly carboxylic acid to the product comprises chemically converting.

5. The method of claim 4, wherein chemically converting is selected from the group consisting of polymerization, condensations, isomerization, esterification, alkylation, oxidation, amination, acid halide formation, reduction, hydrogenation, cyclization, ion exchange, anhydration, acylation and combinations thereof.

6. The method of claim 4, wherein chemically converting includes steps selected from catalytic conversion, non-catalytic conversion and combinations thereof.

7. The method of claim 1, wherein treating is performed initially with one of more enzymes to release one or more sugar from the lignocellulosic or cellulosic material followed by one or more organisms to produce the poly carboxylic acid.

8. The method of claim 7, wherein the sugar is selected from the group consisting of glucose, xylose, sucrose, maltose, lactose, mannose, galactose, arabinose, fructose, disaccharides of any one or two of these, cellobiose, sucrose, poly saccharides of any of two or more of these, and mixtures of these.

9. The method of claim 8, wherein treating converts one or more of the sugars to an intermediate product prior to conversion to the poly carboxylic acid.

10. The method of claim 9, wherein the intermediate product is ethanol or glycol.

11. The method of claim 9, wherein the sugar is converted to the intermediate product by fermentation.

12. The method of claim 1, wherein the poly carboxylic acid is selected from the group consisting of oxalic acid, malonic acid, succinic acid, tartaric acid, glutaric acid, adipic acid, pimelic acid, suberic acid, azelaic acid, sebacic acid, undecanedioic acid, dodecanedioic acid, maleic acid, fumaric acid, glutaconic acid, traumatic acid, muconic acid, phthalic acid, isophthalic acid, terephthalic acid, citric acid, isocitric acid, aconitic acid, mellitic acid and mixtures of these.

13. The method of claim 1, wherein the poly carboxylic acid is succinic acid.

14. The method of claim 13, wherein the product is selected from the group consisting of tetrahydrofuran, gamma-butyro lactone, 2-pyrrolidinone, N-methyl-2-pyrrolidinone (NMP), N-viny-2-pyrrolidinone, succinimide, N-hydroxysuccinimide, succindiamide, succinyl chloride, succinic acid anhydride, maleic anhydride, 1,4-diaminobutane, succinonitrile, 1,4-butandiol and dimethyl succinate

15. A method for making a product comprising:
contacting a mixed sugar solution comprising a nitrogen source and inorganic salts with a succinic acid producing organism to produce succinic acid,
purifying the succinic acid, and
converting the purified succinic acid to the product, wherein
the sugar solution is made by saccharifying an electron beam treated cellulosic or lignocellulosic biomass.

16. The method as in claim 12, wherein the inorganic salts include NaH₂PO₄, Na₂HPO₄, NaCl, MgCl₂ and CaCl₂.

17. The method of claim 15, wherein the nitrogen source includes yeast extract.

18. The method of claim 15, wherein the organism is selected from the group consisting of Actinobacillus succinogenes, Anaerobiospirillum succiniciproducens, Mannheimia succiniciproducens and, PEP carboxylase over-expressing E. coli.

19. The method of claim 15, wherein converting comprises chemically converting.

20. The method of claim 15, wherein the cellulosic or lignocellulosic material receives between about 10 and about 50 Mrad of radiation.

21. A method for making a product comprising:
contacting a mixed sugar solution comprising a nitrogen source and inorganic salts with an organism selected from the group consisting of Actinobacillus succinogenes, Anaerobiospirillum succiniciproducens, Mannheimia succiniciproducens and, PEP carboxylase over-expressing E. coli. and fermenting at least one sugar to succinic acid, and
converting the succinic acid to the product, wherein
the sugar solution is made by saccharifying an electron beam treated cellulosic or lignocellulosic biomass.

22. The method of claim 21, wherein converting comprises chemically converting.

23. The method of claim 22, wherein chemically converting is selected from the group consisting of polymerization, condensations, isomerization, esterification, alkylation, oxidation, amination, acid halide formation, reduction, hydrogenation, cyclization, ion exchange, anhydration, acylation and combinations thereof.

24. The method of claim 21, wherein
lignocellulosic material receives between about 10 and about 50 Mrad of radiation.

25. A method for making a product comprising:
treating a reduced recalcitrance lignocellulosic or cellulosic material with one or more enzymes and/or organisms to produce a poly carboxylic acid.

26. The method of claim 25, wherein a feedstock is pretreated with ionizing radiation to produce the reduced recalcitrance lignocellulosic or cellulosic material.

27. The method of claim 26, wherein the ionizing radiation is performed with an electron beam.

28. The method of claim 25, wherein treating is performed initially with one of more enzymes to release one or more sugar from the lignocellulosic or cellulosic material followed by one or more organisms to produce the poly carboxylic acid.

29. The method of claim 25, wherein the poly carboxylic acid is selected from the group consisting of oxalic acid, malonic acid, succinic acid, tartaric acid, glutaric acid, adipic acid, pimelic acid, suberic acid, azelaic acid, sebacic acid, undecanedioic acid, dodecanedioic acid, maleic acid, fumaric acid, glutaconic acid, traumatic acid, muconic acid, phthalic acid, isophthalic acid, terephthalic acid, citric acid, isocitric acid, aconitic acid, mellitic acid and mixtures of these

30. The method of claim 29, wherein the poly carboxylic acid is not succinic acid.
